(19) United States
(12) Patent Application Publication
(10) Pub. No.: US 2019/0307818 A1
(43) Pub. Date: Oct. 10, 2019

(54) NOVEL ANTIMICROBIAL COMPOSITIONS AND METHODS OF USE

(71) Applicant: The Trustees of Princeton University, Princeton, NJ (US)

(72) Inventors: Bonnie L. Bassler, Princeton (US); Nina M. Hoyland-Kroghsbo, Copenhagen (DK); Jon E. Paczkowski, Princeton (US)

(73) Assignee: The Trustees of Princeton University, Princeton, NJ (US)

(21) Appl. No.: 16/091,990

(22) PCT Filed: Apr. 7, 2017
(86) PCT No.: PCT/US2017/026700

§ 371 Date: Oct. 7, 2018

Related U.S. Application Data
(60) Provisional application No. 62/320,143, filed on Apr. 8, 2016.

(51) Int. Cl. A61K 35/76; A61K 45/06; A61P 31/04; A01N 63/00; G01N 33/68; A61K 31/353; A61K 31/12; A61K 31/44; A61K 31/381; A61K 31/365; A23L 3/3454

(52) U.S. Cl. CPC A61K 35/76; A61K 45/06; A61P 31/04; A01N 63/00; G01N 33/6872; A23V 2002/00; A61K 31/12; A61K 31/44; A61K 31/381; A61K 31/365; A23L 3/3454; A61K 31/353

(57) ABSTRACT

The invention relates to compositions for inhibiting or agonizing quorum sensing (QS) pathways to repress biofilm formation, biofilm streamer formation, virulence factor production, and/or infections (including bacterial infections and bacterial infections with resistance to antibiotics) by: (a) a monotherapy using novel compounds, such as small molecule inhibitors or agonists, to inhibit or activate QS activation pathways and/or (b) a combination therapy including novel compounds to inhibit or activate QS and/or to sensitize bacteria to phage along with phage therapy. These compositions can be used to treat patients having infections. Additionally, these compositions can be used to treat surfaces/areas known to contain human pathogens notorious for causing hospital-acquired infections (such as intravenous catheters, implants, medical devices) as well as fatal infections that occur outside of health care settings. Preferred microorganisms that can be treated with the compositions of the invention include, but are not limited to bacterial strains resistant to one or more antibiotics.

### BACKGROUND

The urgent, unmet need for new classes of antibiotics presents a global challenge to the scientific and medical communities. Traditional methods for anti-microbial development have stagnated while resistance to available antibiotics has skyrocketed. New strategies for combating bacterial infections are needed.

In a process referred to as quorum sensing (QS), microorganisms, such as bacteria, communicate using chemical signaling molecules called autoinducers. QS allows microorganisms to collectively carry out tasks that would be unsuccessful if carried out by individual microorganisms acting alone, thereby utilizing many cells acting in synchrony. QS coordinates collective behaviors among microorganisms in response to fluctuations in population density, and involves the production, release, and detection of extracellular signal molecules called autoinducers. By monitoring increases and decreases in autoinducer concentration, QS bacteria track changes in cell-population density and synchronously switch into and out of group behaviors. QS controlled collective behaviors are induced at high cell density and include bioluminescence, virulence factor production, biofilm formation, the release of public goods, and resistance mechanisms towards bacteriophage (phage) viruses.

Both Gram-positive and Gram-negative infectious bacteria, which include human, animal, plant, and marine pathogens, use QS strategies to control virulence. QS also controls biofilm formation. Biofilms are communities of bacterial cells adhered to surfaces and encased in a self-excreted matrix of extracellular polymeric substances. In most environments, bacteria are found predominantly in biofilms. These biofilms are also widespread in industrial systems and are associated with increased risk of infection when found in clinical environments and in indwelling medical devices. These bacterial biofilm communities can cause chronic infections in humans by colonizing, for example, on or within medical implants, heart valves, or lungs.

In settings involving fluid flow across the biofilm, as in rivers, in industrial manufacturing facilities and medical device systems subject to fluid flow, filamentous biofilms, called streamers, can be formed. These streamers can have a dramatic effect on the biofilm environment. In rivers, for example, the biofilm streamers can increase transient storage and cycling of nutrients and can enhance the retention of suspended particles. In industrial manufacturing facilities, the biofilm streamers have been associated with manufacturing issues such as pipe clogging and pressure drops. In medical devices, biofilm streamers have been associated with device clogging, pressure drops, and chronic infections in humans. Although biofilms and streamers play an important role in industrial, medical and clinical settings, the precise mechanisms driving their formation are poorly understood. QS is, however, known to control streamer formation.

Additionally, existing treatments for bacterial infections using bactericidal or bacteriostatic molecules inhibit biological processes such as cell wall formation, DNA replication, transcription, translation or tetrahydrofolic (THF) acid synthesis. These existing methods for treating bacterial infections unfortunately exacerbate the growing antibiotic resistance problem because they inherently select for survival of bacteria that can resist the drug.

For example, Staphylococcus aureus is a human pathogen notorious for causing hospital-acquired infections as well as fatal infections that occur outside of health care settings. S. aureus infections that are associated with abiotic materials, such as intravenous catheters, intubation equipment, and implants, are of primary concern as S. aureus readily colonizes such medical devices, forming biofilms, biofilm streamers and initiating virulence factor production under these conditions.

In fact, methicillin-resistant S. aureus (MRSA) is a major concern due to its potent virulence coupled with resistance to many antibiotics. MRSA is a leading cause of hospital-associated infections in the United States and Europe with a high mortality rate. S. aureus and MRSA cause a variety of infections ranging from minor skin infections to serious illnesses such as infections of indwelling medical devices, osteomyelitis, endocarditis, sepsis, and toxic shock syndrome. S. aureus is just one example of a microorganism that uses QS mediated communication to control virulence factor production and to regulate biofilm formation.

As another example, the bacterium Pseudomonas aeruginosa is the major pathogen associated with cystic fibrosis lung infection, hospital acquired infections, urinary tract infections, keratitis eye infection, and third-degree burn-associated skin infections. Some strains of P. aeruginosa are resistant to antibiotic therapy. P. aeruginosa has a complex signaling pathway that governs QS and virulence. The signaling pathway includes LasI, a synthase enzyme that produces native acyl-homoserine lactone (AHL) autoinducer signal, 3-O—C₁₂-HSL. The native autoinducer signal is detected by the transcriptional regulator LasR, forming a LasR:3-O—C₁₂-HSL complex. The LasR:3-O—C₁₂-HSL complex affects gene transcription, by turning on virulence factors, biofilm genes and the rhl QS system and additional QS circuits.

Another synthase, RhlI, produces the acyl-homoserine lactone (AHL) autoinducer, N-butyryl-L-homoserine lactone C₄-HSL, which is detected by the transcriptional regulator RhlR. The RhlR:C₄-HSL complex also regulates virulence genes, biofilm genes, and other components of the signaling pathway. Virulence production is impacted by multiple other factors, including the transcription factor QscR and the PQS QS system that produces and detects quinolone autoinducer signals.

This tandem regulatory arrangement allows LasI/R to control the first wave of QS controlled gene expression and RhlI/R to control the second. Because LasR activates expression of rhlR, deletion of lasR reduces expression of both lasR- and rhlR-regulated target genes.

Additionally, one key factor in pathogenicity of a bacterial infection is the production of virulence factor produced at high cell density, such as pyocyanin. This small molecule is redox active and is important for maintaining the redox balance in P. aeruginosa, particularly under low oxygen or anaerobic conditions. RhlR is a key transcriptional regulator controlling the up-regulation of the pyocyanin biosynthetic pathway, which in turn is induced by the LasR:3-O—C₁₂-HSL complex.

Thus, what is needed are novel methods of inhibiting or activating QS to repress biofilm formation, biofilm streamer formation, virulence factor production, and/or infections (including bacterial infections and bacterial infections resistant to antibiotics) by (a) a monotherapy using novel compounds, such as small molecule inhibitors/agonists, to inhibit/activate QS pathways and/or (b) a combination therapy including novel compounds to inhibit/activate QS along with phage therapy.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

The present invention targets the quorum sensing (“QS”) mechanism undertaken by microorganisms, which is crucial in microbial infection and pathogenicity. Present invention embodiments include a novel approach to inhibit QS, biofilm formation, biofilm streamer formation, virulence factor production, and/or infections (including bacterial infections and bacterial infections resistant to antibiotics) by: (a) a monotherapy using novel compounds, such as small molecule inhibitors, to inhibit QS activation pathways; and/or (b) a combination therapy including novel compounds to inhibit QS along with phage therapy. Administration of QS inhibitors (QS antagonists), or a combination of QS inhibitors and phage therapy, results in significant reductions in biofilm formation and virulence factor production of microbial organisms, and can be used to treat infections (including bacterial infections and bacterial infections resistant to antibiotics).

Thus, present invention embodiments relate to a method of inhibiting QS, biofilm production, biofilm streamer production, virulence factor production and/or infections (including bacterial infections and bacterial infections resistant to antibiotics) by a microorganism using: (1) a small molecule antagonist to decrease or inhibit QS, biofilm production, biofilm streamer production, virulence factor production and/or infections (including bacterial infections and bacterial infections resistant to antibiotics) by a microorganism, and/or (2) a combination therapy of a small molecule antagonist and phage therapy to decrease or inhibit QS, phage resistance, biofilm production, biofilm streamer production, virulence factor production and/or infections (including bacterial infections and bacterial infections resistant to antibiotics) by a microorganism.

In other aspects, present invention embodiments relate to a method of agonizing QS pathways to repress biofilm production, biofilm streamer production, virulence factor production and/or infections (including bacterial infections and bacterial infections resistant to antibiotics such as cholera) by a microorganism using: (1) a small molecule agonist to activate QS to repress biofilm production, biofilm streamer production, virulence factor production and/or infections (including bacterial infections and bacterial infections resistant to antibiotics) by a microorganism, and/or (2) a combination therapy of a small molecule agonist and phage therapy to activate QS to repress biofilm production, biofilm streamer production, virulence factor production and/or infections (including bacterial infections and bacterial infections resistant to antibiotics) by a microorganism.

According to embodiments of the invention, QS inhibitors/agonists may be administered to a patient to treat or prevent a bacterial infection. In some aspects, QS inhibitors may include flavonoid type compounds (e.g., quercetin, baicalein, etc.) that target LasR and RhlR, master regulators of the QS pathway in P. aeruginosa. QS inhibitors/agonists block virulence and the formation of normal biofilms. Additionally, in bacteria that utilize a CRISPR-Cas immune system, blocking QS with a QS inhibitor/agonist suppresses the CRISPR-Cas system, thus blocking immunity and adaptation mechanisms that a bacterial cell utilizes for evading phage therapy, and thus, sensitizing the bacterial cell to infection by natural or engineered phages.

According to other embodiments of the invention, combination therapies that utilize a QS inhibitor/agonist coupled with other non-traditional therapies, such as phage therapies, may be administered to a patient to treat or prevent antibiotic resistant infections.

As used herein, a biofilm, a biofilm streamer, and/or a virulence factor are produced or formed by a microorganism(s). In preferred embodiments, the microorganism is selected from the following groups: bacteria and/or archaea. In further embodiments, the bacteria and/or archaea are pathogenic to humans, animals and/or plants. In further embodiments the bacteria and/or archaea are common to industrial settings, such as, for example, industrial fluid handling processes, pharmaceutical manufacturing processes, medical processes or devices, agricultural processes, and/or machinery. In further embodiments, the bacteria and/or archaea are common to an apparatus and/or process that involves fluid flow.

In still further embodiments the bacteria are selected from the following genera: Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anabaena, Anabaenopsis, Anaerobospirillum, Anaerorhabdus, Aphanizomenon, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Bordetella, Borrelia, Brachyspira, Branhamella, Brevibacillus, Brevibacterium, Brevundimonas, Brucella, Burkholderia, Buttiauxella, Butyrivibrio, Calymmatobacterium, Camesiphon, Campylobacter, Capnocytophaga, Capnylophaga, Cardiobacterium, Catonella, Cedecea, Cellulomonas, Centipeda, Chlamydia, Chlamydophila, Chromobacterium, Chryseomonas, Chyseobacterium, Citrobacter, Clostridium, Collinsella, Comamonas, Corynebacterium, Coxiella, Cryptobacterium, Cyanobacteria, Cylindrospermopsis, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gloeobacter, Gordona, Haemophilus, Hafnia, Hapalosiphon, Helicobacter, Helococcus, Hemophilus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptospirae, Leptotrichia, Leuconostoc, Listeria, Listonella, Lyngbya, Megasphaera, Methylobacterium, Microbacterium, Micrococcus, Microcystis, Mitsuokella, Mobiluncus, Moellerella, Moraxella, Morganella, Mycobacterium, Mycoplasma, Myroides, Neisseria, Nocardia, Nocardiopsis, Nodularia, Nostoc, Ochrobactrum, Oeskovia, Oligella, Orientia, Paenibacillus, Pantoea, Parachlamydia, Pasteurella, Pediococcus, Peptococcus, Peptostreptococcus, Phormidium, Photobacterium, Photorhabdus, Phyllobacterium, Phytoplasma, Planktothrix, Plesiomonas, Porphyromonas, Prevotella, Propionibacterium, Proteus, Providencia, Pseudoanabaena, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Schizothrix, Selenomonas, Serpulina, Serratia, Shewenella, Shigella, Simkania, Slackia, Sphaerotilus, Sphingobacterium, Sphingomonas, Spirillum, Spiroplasma, Spirulina, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Trichodesmium, Tropheryma, Tsakamurella, Turicella, Umezakia, Ureaplasma, Vagococcus, Veillonella, Vibrio, Weeksella, Wolinella, Xanthomonas, Xenorhabdus, Yersinia, and Yokenella.

In still further embodiments the bacteria are selected from the following species: Acinetobacter baumannii, Actinobacillus actinomycetemcomitans, Actinobacillus pleuropneumoniae, Actinomyces bovis, Actinomyces israelii, Bacillus anthracis, Bacillus ceretus, Bacillus coagulans, Bacillus liquefaciens, Bacillus popillae, Bacillus subtilis, Bacillus thuringiensis, Bacteroides distasonis, Bacteroides fragilis, Bacteroides thetaiotaomicron, Bacteroides vulgatus, Bartonella bacilliformis, Bartonella Quintana, Beneckea parahaemolytica, Bordetella bronchiseptica, Bordetella parapertussis, Bordetella pertussis, Borelia burgdorferi, Brevibacterium lactofermentum, Brucella abortus, Brucella canis, Brucella melitensis, Brucella suis, Burkholderia cepacia, Burkholderia mallei, Burkholderia pseudomallei, Campylobacter fetus, Campylobacterjejuni, Campylobacter pylori, Cardiobacterium hominis, Chlamydiapneumoniae, Chlamydia psittaci, Chlamydia trachomatis, Chlamydophila abortus, Chlamydophila caviae, Chlamydophila felis, Chlamydophila pneumonia, Chlamydophila psittaci, Chryseobacterium eningosepticum, Clostridium botulinum, Clostridium butyricum, Clostridium coccoides, Clostridium dijficile, Clostridium leptum, Clostridium tetani, Corynebacterium xerosis, Cowdria ruminantium, Coxiella burnetii, Edwardsiella tarda, Ehrlichia sennetsu, Eikenella corrodens, Elizabethkingia meningoseptica, Enterobacter aerogenes, Enterobacter cloacae, Enterococcus faecalis, Escherichia coli, Escherichia hirae, Flavobacterium meningosepticum, Fluoribacter bozemanae, Francisella tularensis, Francisella tularensis biovar Tularensis, Francisella tularensis subsp. Holarctica, Francisella tularensis subsp. nearctica, Francisella tularensis subsp. Tularensis, Francisella tularensis var. palaearctica, Fudobascterium nucleatum, Fusobacterium necrophorum, Haemophilus ducreyi, Haemophilus influenzae, Helicobacter pylori, Kingella kingae, Klebsiella mobilis, Klebsiella oxytoca, Klebsiella pneumoniae, Lactobacillus acidophilus, Lactobacillus casei, Lactobacillus hilgardii, Lactobacillus pentosus, Lactobacillus plantarum, Lactobacillus rhamnosus, Lactococcus lactis, Legionella bozemanae corrig., Legionella pneumophila, Leptospira alexanderi, Leptospira borgpetersenii, Leptospira fainei, Leptospira inadai, Leptospira interrogans, Leptospira kirschneri, Leptospira noguchii, Leptospira santarosai, Leptospira weilii, Leuconostoc lactis, Leuconostoc oenos, Listeria ivanovii, Listeria monocytogenes, Moraxella catarrhalis, Morganella morganii, Mycobacterium africanum, Mycobacterium avium, Mycobacterium avium subspecies paratuberculosis, Mycobacterium bovis, Mycobacterium bovis strain BCG, Mycobacterium intracellulare, Mycobacterium kansasii, Mycobacterium leprae, Mycobacterium marinum, Mycobacterium tuberculosis, Mycobacterium typhimurium, Mycobacterium ulcerans, Mycoplasma hominis, Mycoplasma mycoides, Mycoplasma pneumoniae, Neisseria gonorrhoeae, Neisseria meningitidis, Neorickettsia sennetsu, Nocardia asteroides, Orientia tsutsugamushi, Pasteurella haemolytica, Pasteurella multocida, Plesiomonas shigelloides, Propionibacterium acnes, Proteus mirabilis, Proteus morganii, Proteus penneri, Proteus rettgeri, Proteus vulgaris, Providencia alcalifaciens, Providencia rettgeri, Pseudomonas aeruginosa, Pseudomonas mallei, Pseudomonas pseudomallei, Pyrococcus abyssi, Rickettsia akari, Rickettsia canadensis, Rickettsia canadensis corrig, Rickettsia conorii, Rickettsia montanensis, Rickettsia montanensis corrig, Rickettsia prowazekii, Rickettsia rickettsii, Rickettsia sennetsu, Rickettsia tsutsugamushi, Rickettsia typhi, Rochalimaea quintana, Salmonella arizonae, Salmonella choleraesuis subsp. arizonae, Salmonella enterica subsp. Arizonae, Salmonella enteritidis, Salmonella paratyphi, Salmonella typhi, Salmonella typhimurium, Selenomonas nominantium, Selenomonas ruminatium, Serratia marcescens, Shigella boydii, Shigella dysenteriae, Shigella flexneri, Shigella sonnei, Spirillum minus, Staphylococcus aureus, Staphylococcus epidermidis, Staphylococcus equi, Staphylococcus lugdunensis, Stenotrophomonas maltophila, Streptobacillus moniliformis, Streptococcus agalactiae, Streptococcus bovis, Streptococcus ferus, Streptococcus pneumoniae, Streptococcus pyogenes, Streptococcus viridans, Streptomyces ghanaenis, Streptomyces hygroscopicus, Streptomyces phaechromogenes, Treponema carateum, Treponema denticola, Treponema pallidum, Treponema pertenue, Vibrio cholerae, Vibrio parahaemolyticus, Vibrio vulnificus, Xanthomonas maltophilia, Yersinia enterocolitica, Yersinia pestis, Yersinia pseudotuberculosis, and Zymomonas mobilis.

In still further embodiments, the bacteria are from the class of bacteria known as Fusospirochetes.

Such pathogenic bacteria can cause bacterial infections and disorders related to infections that include, but are not limited to, the following: acne, rosacea, skin infection, pneumonia, otitis media, sinusitus, bronchitis, tonsillitis, and mastoiditis related to infection by Streptococcus pneumoniae, Haemophilus influenzae, Moraxella catarrhalis, Staphylococcus aureus, Peptostreptococcus spp. or Pseudomonas spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydiapneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), S. pyogenes, S. agalactiae, Streptococcal groups C-F (minute-colony streptococci), viridans streptococci, Corynebacterium spp., Clostridium spp., or Bartonella henselae; uncomplicated acute urinary tract infections related to infection by S. saprophyticus or Enterococcus spp.; urethritis and cervicitis; sexually transmitted diseases related to infection by Chlamydia trachomatis, Haemophilus ducreyi, Treponema pallidum, Ureaplasma urealyticum, or Nesseria gonorrheae; toxin diseases related to infection by S. aureus (food poisoning and Toxic shock syndrome), or Groups A, S, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by C. trachomatis, N. gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; odontogenic infection related to infection by viridans streptococci; persistent cough related to infection by Bordetella pertussis; gas gangrene related to infection by Clostridium perfringens or Bacteroides spp.; skin infection by S. aureus, Propionibacterium acne; atherosclerosis related to infection by Helicobacter pylori or Chlamydia pneumoniae; or the like. The QS inhibitors/agonists or a combination of the QS inhibitors/agonists and phage therapy as described herein can be used to treat any of these disorders.

In further preferred examples, pathogenic bacteria harbouring a CRISPR-Cas system are selected from the group consisting of, but not limited to: Bacillus anthracis, Campylobacter jejuni, Clostridium botulinum, Clostridium diffcile, Corynebacterium diphtheria, Corynebacterium pseudotuberculosis, Escherichia coli, Enterococcus faecalis, Erwinia amylovora, Francisella cf novicida, Gardnerella vaginalis, Helicobacter pylori, Klebsiella pneumoniae, Legionella pneumophila, Leptospira interrogans, Listeria monocytogenes, Mannheimia haemolytica, Mycobacterium bovis, Mycobacterium tuberculosis, Mycoplasma gallisepticum, Mycoplasma pneumoniae, Neisseria meningitides, Pseudomonas aeruginosa, Salmonella enterica, Shigella flexneri, Staphylococcus epidermidis, Staphylococcus aureus, Streptococcus pyogenes, Vibrio cholera, Yersinia pseudotuberculosis, and Yersinia pestis.

In certain embodiments the disease or disorder that can be treated with QS inhibitors/agonists or a combination of QS inhibitors/agonists and phage therapy as described herein include sepsis, pneumonia, otitis media, chronic obstructive pulmonary disease, urinary tract infections, infections associated with burns, and/or any combinations thereof. In preferred embodiments, infections caused by P. aeruginosa, including infections associated with cystic fibrosis or burns as well as urinary tract infections may be treated with QS inhibitors and/or phage therapy.

In still further embodiments, QS inhibitors/agonists or a combination of QS inhibitors/agonists and phage therapy as described herein can be used to treat a periodontal disease, such as gingivitis, periodontitis or breath malodor.

In still further embodiments, QS inhibitors/agonists or a combination of QS inhibitors/agonists and phage therapy as described herein can be used to treat infections, including but not limited to those infections caused by bacteria. In some embodiments, the bacteria are Gram-negative or Gram-positive bacteria. Non-limiting examples of diseases and/or disorders that can be treated and/or prevented with QS inhibitors/agonists or a combination of QS inhibitors/agonists and phage therapy include otitis media, prostatitis, cystitis, bronchiectasis, bacterial endocarditis, osteomyelitis, dental caries, periodontal disease, infectious kidney stones, acne, Legionnaire's disease, chronic obstructive pulmonary disease (COPD), and cystic fibrosis.

In one specific example, subjects with cystic fibrosis can display with an accumulation of biofilm in the lungs and digestive tract. Subjects afflicted with COPD, such as emphysema and chronic bronchitis, display a characteristic inflammation of the airways wherein airflow through such airways, and subsequently out of the lungs, is chronically obstructed. Infections, including biofilm-related disorders, also encompass infections on implanted or inserted medical devices, such as infections from biliary stents, orthopedic implant infections, and catheter-related infections (e.g., kidney, vascular, peritoneal, etc.). An infection can also originate from sites where the integrity of the skin and/or soft tissue has been compromised. Non-limiting examples include dermatitis, ulcers from peripheral vascular disease, a burn injury, and trauma. All of these diseases and/or disorders can be treated using the QS inhibitors/agonists or a combination of the QS inhibitors/agonists and phage therapy as described herein.

In some embodiments, the QS inhibitors/agonists or a combination of the QS inhibitors/agonists and phage therapy can be administered to a patient via any route involving the gastrointestinal tract, but may alternatively be administered using other known routes of administration including parenteral, for example, by injection, including subcutaneous, intradermal, transdermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. Other convenient routes of administration are not excluded.

In other embodiments, the QS inhibitors/agonists or the combination of QS inhibitors/agonists and phage therapy can be administered to a patient topically, orally, sublingually or via a respiratory route (e.g., inhalation). For example, the QS inhibitors/agonists or the combination of QS inhibitors/agonists and phage therapy is administered in the form of a powder, mist, vapor or any equivalent that may be administered via inhalation into the lungs to treat patients with a respiratory infection. As another example, the QS inhibitors/agonists or the combination of QS inhibitors/agonists and phage therapy is administered in the form of a gel, spray, ointment or other topical equivalent for treatment of bacterial skin infections, to impede the entry of pathogenic bacteria at wound sites, to prevent skin infections in burn victims, or treat other types of surface infections. As yet another example, the QS inhibitors/agonists or the combination of QS inhibitors/agonists and phage therapy is administered orally in the form of a capsule, gel, tablet, liquid or other equivalent for treatment of gastrointestinal infections or other infections.

Present invention embodiments can be also administered in the form of a topical gel, spray, solution, cleansing wipe to inhibit microorganism or bacterial growth, decontaminate, disinfect or sterilize surfaces (e.g., medical device surfaces, manufacturing surfaces, etc.) or other objects (e.g., clothing contaminated with bacteria or microorganisms, etc.). Present invention embodiments can also be utilized to disinfect clothing worn by physicians or patients (e.g., as part of a laundering or cleaning process). In other embodiments, the compositions provided herein can be embedded in a material formulated for slow release (e.g., such as from a medical device, or other material implanted within the patient that slowly dissolves).

Present invention embodiments can be immediately applied to numerous current and urgent issues in healthcare and manufacturing settings to sterilize equipment or provide sterile environments. By administering the compositions described herein as a topical treatment for disinfecting medical or surgical instrumentation (e.g., catheters, colonoscopy equipment, etc.), as a disinfectant to indwelling medical devices (e.g., pacemakers, implantable pain management devices, implantable devices to regulate blood sugar, etc.) prior to implantation to prevent or reduce the accidental introduction of pathogens into patients during medical procedures to prevent an infection caused by a contaminated medical device.

In still other embodiments, the techniques and compositions provided herein may be used as a replacement for bacteriocide. Agricultural produce may be sprayed with or submerged in a solution comprising the QS inhibitors/agonists and/or phage therapies described herein in order to prevent or inhibit colonization of microorganisms including bacteria or contamination with microorganisms of food intended for human or animal consumption (e.g., animal production feed, veterinary medicine, etc.).

In still other embodiments, the techniques and compositions provided herein may be used in animal husbandry, to prevent or treat bacterial or microorganism infections in animals (e.g., cows, chickens, sheep, pigs, fish, or any other domesticated or wild animal). Administration of the compositions may be via an oral route or any other suitable route of administration, including the routes of administration described herein.

In still other embodiments, the techniques and compositions provided herein may be used in the food industry to prevent microorganism contamination including bacterial contamination of both plant and animal based food products during harvesting, as well as promote food safety, by inhibiting microorganism growth and contamination in animal and plant food processing, handling, packaging, preservation and production. In still other embodiments, the QS inhibitor/agonist and/or phage therapies can be applied as a coating on a ship as an “antifouling” agent to prevent biofilm growth on the bottom of the ship.

The present invention also relates to a method of screening a test compound that can inhibit (i.e. reduce or inhibit) QS, biofilm formation, biofilm streamer formation, virulence factor production and/or infections (including bacterial infections and bacterial infections resistant to antibiotics) by a microorganism by contacting the microbial organism with a QS inhibitor (i.e., an antagonist), and measuring the reduction or elimination of QS, biofilm formation, biofilm streamer formation, virulence factor production, and/or infection after treatment. The method also includes screening a composition comprising a test compound and one or more particular phage therapies that can inhibit (i.e. reduce or inhibit) QS, biofilm formation, biofilm streamer formation, virulence factor production and/or infections (including bacterial infections and bacterial infections resistant to antibiotics) by a microorganism by contacting the microbial organism with a QS inhibitor (i.e. an antagonist) and phage therapy, and measuring the reduction or elimination of QS, biofilm formation, biofilm streamer formation, virulence factor production, and/or infection after treatment. Other embodiments that include a method of screening compounds or compositions as described herein that can inhibit infections by microorganisms are also contemplated.

Still other embodiments include screening a test compound that can activate (agonize) QS to repress biofilm formation, biofilm streamer formation, virulence factor production and/or infections (including bacterial infections and bacterial infections resistant to antibiotics) by a microorganism by contacting the microbial organism with a QS agonist and/or phage therapy, and measuring the activation of QS and corresponding inhibition of biofilm formation, biofilm streamer formation, virulence factor production, and/or infection after treatment.

### BRIEF DESCRIPTION OF THE DRAWINGS

Embodiments are illustrated by way of example and are not limited to the figures of the accompanying drawings, in which like references indicate similar elements and in which:

FIG. 1. QS molecules regulate cas3 expression. Quantitative Real Time-PCR (qRT-PCR) was utilized to determine the relative expression of cas3, and the expression profiles were normalized based on expression of 5S ribosomal RNA. Cas3 encodes a nuclease and helicase that degrades foreign DNA, such as phage DNA, plasmid DNA, transposon DNA, etc. Expression data included wild type (WT) Pseudomonas aeruginosa PA14, QS mutants (ΔlasR, ΔrhlR, ΔlasR ΔrhlR and ΔlasI ΔrhII) grown in the presence of DMSO solvent (as a control), and QS mutant (ΔlasI ΔrhII) grown in the presence of autoinducers (AIs), including 100 uM 3-O—C₁₂-HSL and 100 uM C₄-HSL. AIs 3-O—C₁₂-HSL and C₄—HSL rescue cas3 expression in QS deficient mutants. Thus, molecules involved in QS (e.g., the expression products of lasR, rhlR, lasI, and rhII) were shown to regulate the expression of the cas3 genes.

FIG. 2A Genetic depletion of molecules involved in QS downregulates immune activity conferred by the CRISPR-Cas system. The efficiency of transformation (EOT), which involves the introduction of foreign DNA (e.g., a plasmid comprising a small sequence targeted for elimination by the CRISPR-Cas system), was determined for wild type bacterial strain P. aeruginosa PA14, CRISPR-Cas deficient strain (ΔCRISPR Δcas), QS deficient strain (ΔlasI ΔrhII), and QS deficient strain (ΔlasI ΔrhII) rescued by AIs 3-O—C₁₂-HSL and C₄—HSL. In the WT PA14 strain, the efficiency of transformation was determined to be 0.7%, indicating that the bacterial immune system was 99.3% efficient at eliminating foreign DNA via the CRISPR-Cas system. In the absence of CRISPR-Cas (the ΔCRISPR Δcas strain), the bacterium failed to eliminate the foreign DNA. QS mutant (ΔlasI ΔrhII) had more than a 10-fold increase in EOT as compared to WT, indicating that this QS mutant was more than 10-fold less efficient in eliminating foreign DNA. Adding 3-O—C₁₂-HSL and C₄—HSL (AIs) to the QS mutant (ΔlasI ΔrhII) restored immune activity to nearly WT levels.

FIG. 2B Genetic depletion of molecules involved in QS downregulates immune activity conferred by the CRISPR-Cas system towards phage virus. The efficiency of plaquing, which is a measure of the ability of phage virus to kill bacteria, was determined for wild type bacterial strain P. aeruginosa PA14 and QS deficient strain (ΔlasI ΔrhII ΔpqsA). The PA14 strain had been adapted to phage JBD44a, and a single CRISPR spacer was confirmed in the CRISPR2 locus. This adapted strain was used to generate the adapted QS mutant strain (ΔlasI ΔrhII ΔpqsA). A virulent variant of JBD44a, JBD44a^{vir}, was spotted in five-fold dilutions on soft agar overlays of the adapted strains PA14 and ΔlasI ΔrhII ΔpqsA. The plaquing efficiency of BD44a^{vir} was approximately 10-fold higher on the QS mutant (ΔlasI ΔrhII ΔpqsA) than the WT, indicating that the QS mutant was 10-fold less efficient in surviving a phage virus attack.

FIG. 3. QS inhibitors block CRISPR adaptation and disable QS-mediated bacterial immunity. A P. aeruginosa PA14 ΔlasI ΔrhII QS deficient strain was transformed with foreign DNA (e.g., a CRISPR-targeted plasmid). A single colony from the transformation was re-streaked on selective media containing either DMSO (as a control), AIs (2 μM 3-O—C₁₂-HSL+10 μM C₄-HSL), or AIs and a QS inhibitor (2 μM 3-O—C₁₂-HSL+10 μM C₄-HSL+100 μM Baicalein). Single colonies were tested for integration of new inserted spacers (conferring immunity) against the CRISPR-targeted plasmid. In particular, PCR primers were designed to amplify specific genomic regions into which spacers were inserted. Primers complementary to the region upstream of the CRISPR region and to the second spacer region were designed, and PCR amplification was utilized to test for the presence of the newly inserted spacer (a process referred to as adaptation). As compared to the DMSO control, addition of AIs increased the fraction of cells in the colony that adapted, while addition of AIs in combination with a QS inhibitor (e.g., Baicalein) blocked the positive effects of AIs on CRISPR adaptation.

FIG. 4. QS inhibitors bind non-competitively to LasR. A 10-point dose response curve with increasing concentrations of autoinducer 3-O—C₁₂-HSL (AI) was generated for a variety of QS small molecule inhibitors. In this figure, curve C12 (filled circle) corresponds to the absence of inhibitor. The other lines (triangular, diamond, and open circle) correspond to the presence of a QS inhibitor (at 100 μM), i.e. compound #1 corresponds to phloretin, compound #3 corresponds to chrysin, compound #46 corresponds to baicalein, and compound #48 corresponds to quercetin, which were each found to inhibit both LasR and RhlR. Despite the increasing concentration of AIs, LasR-dependent transcription of the reporter remained significantly inhibited at the highest dose, indicating that the inhibitors did not compete (bind to the same ligand-binding pocket) with the AI. Instead, the inhibitors bind elsewhere on LasR to prevent LasR-dependent transcription.

FIG. 5. QS inhibitors block gene transcription by preventing receptor-DNA binding. A DNA-binding assay (an electrophoretic mobility shift assay) was performed to assess the DNA-binding ability of LasR complexed with an inhibitor (e.g., compound #1, compound #3, or compound #46). Recombinantly expressed and purified LasR protein bound to the AI (3-O—C₁₂-HSL) was incubated with DMSO (as a control) and 100 μM of the inhibitor for 15 min, and then mixed with a radioactively labeled LasR-dependent promoter DNA sequence for 30 min. Three different concentrations of LasR were used for each experiment (0 nM, 12.5 nM, and 25 nM). Each of the small molecule QS inhibitors was shown to reduce the affinity of LasR for DNA by at least 50%, indicating that these molecules blocked gene transcription by preventing LasR from binding to DNA.

FIG. 6. QS inhibitors reduce the level of virulence factor production in P. aeruginosa. The ability of the small molecule inhibitors to reduce virulence factor production by monitoring pyocyanin production was assessed. Wild-type P. aeruginosa was incubated overnight with the indicated molecules at 100 μM, back diluted, and grown for 18 hrs in the presence of an inhibitor. mBTL was previously shown to inhibit pyocyanin production. As pyocyanin production is controlled by the LasR-RhlR QS system (a ΔlasR ΔrhlR mutant does not produce pyocyanin), inhibiting either of the receptors should result in decreased production of the virulence factor. Interestingly, these compounds (compounds #1, #3, #4, #46 and #48) also inhibit pyocyanin to varying degrees, likely dependent on their ability to bypass metabolism and/or efflux pumps in P. aeruginosa.

FIGS. 7A and 7B. Plasmid maps are shown for the two plasmid E. coli reporter system. Experimental protocol is provided in Example 5.

FIG. 8. Dose dependence of QS inhibitors. QS inhibitors (e.g., flavonoids) were also shown to inhibit pyocyanin in a dose-dependent manner.

FIGS. 9A-B. The ΔrhlI mutant was virulent and the ΔrhlR mutant was avirulent in animal infection models. (9A) Bacterial load in lung homogenate of mice infected intratracheally with WT P. aeruginosa UCBPP-PA14, and the ΔrhlR and ΔrhII mutants was determined. Each symbol in FIG. 9A represents a single mouse. The data were pooled from two independent experiments. Data were analyzed using the Mann-Whitney U test. *** P<0.001 and ns means not significant. (9B) Real-time monitoring of WT P1-lux and isogenic mutants in the acute pneumonia model was performed. BALB/c mice infected with WT, and the ΔrhlR and ΔrhII strains were imaged at 24 and 48 h using an IVIS CCD camera following intratracheal infection. Imaging was performed from the ventral side while the animals were under isoflourane anesthesia. The color bar indicates the intensity of the bioluminescence output, with red and blue denoting the high and low signals, respectively. Note that the color scales on the various mouse bioluminescence imaging panels are not the same.

FIGS. 10A-B. The ΔlasI and ΔlasR mutants exhibited avirulent phenotypes in rodent infection models. (10A) Bacterial load in lung homogenate of mice infected intratracheally with WT P. aeruginosa UCBPP-PA14 and the ΔlasR and ΔlasI mutants was determined. Each symbol represents a single mouse. The data were pooled from two independent experiments. Data were analyzed using the Mann-Whitney U test. *** P<0.001 and ns means not significant. (10B) Real-time monitoring of WT P1-lux and isogenic mutants in the acute pneumonia model. BALB/c mice infected intratracheally with WT, and the ΔlasR, and ΔlasI strains were imaged at 24 and 48 h using an IVIS CCD camera. Imaging was performed from the ventral side of representation mice while the animals were under isoflourane anesthesia. Results for representative mice are shown. The color bar indicates the intensity of the bioluminescence output, with red and blue denoting the high and low signals, respectively. Note that the color scales on the various mouse bioluminescence imaging panels are not the same.

### DETAILED DESCRIPTION

### A. Definitions

Unless defined otherwise, all technical and scientific terms used herein have the same meaning as commonly understood by those of ordinary skill in the art, such as in the arts of microbiology, peptide chemistry, cell culture, nucleic acid chemistry, immunology and biochemistry. Standard techniques are used for molecular biology, genetic and biochemical methods (see Sambrook et al., Molecular Cloning: A Laboratory Manual, 3^{rd} ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

As used herein, a “microorganism” is defined as a bacterium and/or an archaeon.

As used herein, “bacteria” are defined as any one of a large domain of single-celled prokaryotic microorganisms. As used herein, bacteria include any that are known to those of ordinary skill in the art and any that may be discovered. Preferred examples of bacteria are those known to be pathogenic to humans, animals or plants. Other preferred examples include those known to cause undesirable contamination and/or clogging of industrial fluid flow systems. Still other preferred examples of bacteria include those known to contaminate or impede the function of implanted medical devices (pumps, stents, artificial joints, screws, rods, and the like). Further preferred examples of bacteria include those capable of forming biofilms and/or biostreamers or producing virulence factors. Further preferred examples include bacteria selected from the following genera: Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anabaena, Anabaenopsis, Anaerobospirillum, Anaerorhabdus, Aphanizomenon, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Bordetella, Borrelia, Brachyspira, Branhamella, Brevibacillus, Brevibacterium, Brevundimonas, Brucella, Burkholderia, Buttiauxella, Butyrivibrio, Calymmatobacterium, Camesiphon, Campylobacter, Capnocytophaga, Capnylophaga, Cardiobacterium, Catonella, Cedecea, Cellulomonas, Centipeda, Chlamydia, Chlamydophila, Chromobacterium, Chryseomonas, Chyseobacterium, Citrobacter, Clostridium, Collinsella, Comamonas, Corynebacterium, Coxiella, Cryptobacterium, Cyanobacteria, Cylindrospermopsis, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gloeobacter, Gordona, Haemophilus, Hafnia, Hapalosiphon, Helicobacter, Helococcus, Hemophilus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptospirae, Leptotrichia, Leuconostoc, Listeria, Listonella, Lyngbya, Megasphaera, Methylobacterium, Microbacterium, Micrococcus, Microcystis, Mitsuokella, Mobiluncus, Moellerella, Moraxella, Morganella, Mycobacterium, Mycoplasma, Myroides, Neisseria, Nocardia, Nocardiopsis, Nodularia, Nostoc, Ochrobactrum, Oeskovia, Oligella, Orientia, Paenibacillus, Pantoea, Parachlamydia, Pasteurella, Pediococcus, Peptococcus, Peptostreptococcus, Phormidium, Photobacterium, Photorhabdus, Phyllobacterium, Phytoplasma, Planktothrix, Plesiomonas, Porphyromonas, Prevotella, Propionibacterium, Proteus, Providencia, Pseudoanabaena, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Schizothrix, Selenomonas, Serpulina, Serratia, Shewenella, Shigella, Simkania, Slackia, Sphaerotilus, Sphingobacterium, Sphingomonas, Spirillum, Spiroplasma, Spirulina, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Trichodesmium, Tropheryma, Tsakamurella, Turicella, Umezakia, Ureaplasma, Vagococcus, Veillonella, Vibrio, Weeksella, Wolinella, Xanthomonas, Xenorhabdus, Yersinia, and Yokenella.

Further preferred examples include bacteria selected from the following species: Acinetobacter baumannii, Actinobacillus actinomycetemcomitans, Actinobacillus pleuropneumoniae, Actinomyces bovis, Actinomyces israelii, Bacillus anthracis, Bacillus ceretus, Bacillus coagulans, Bacillus liquefaciens, Bacillus popillae, Bacillus subtilis, Bacillus thuringiensis, Bacteroides distasonis, Bacteroides fragilis, Bacteroides thetaiotaomicron, Bacteroides vulgatus, Bartonella bacilliformis, Bartonella Quintana, Beneckea parahaemolytica, Bordetella bronchiseptica, Bordetella parapertussis, Bordetella pertussis, Borelia burgdorferi, Brevibacterium lactofermentum, Brucella abortus, Brucella canis, Brucella melitensis, Brucella suis, Burkholderia cepacia, Burkholderia mallei, Burkholderia pseudomallei, Campylobacter fetus, Campylobacter jejuni, Campylobacter pylori, Cardiobacterium hominis, Chlamydia pneumoniae, Chlamydia psittaci, Chlamydia trachomatis, Chlamydophila abortus, Chlamydophila caviae, Chlamydophila felis, Chlamydophila pneumonia, Chlamydophila psittaci, Chryseobacterium eningosepticum, Clostridium botulinum, Clostridium butyricum, Clostridium coccoides, Clostridium dijficile, Clostridium leptum, Clostridium tetani, Corynebacterium xerosis, Cowdria ruminantium, Coxiella burnetii, Edwardsiella tarda, Ehrlichia sennetsu, Eikenella corrodens, Elizabethkingia meningoseptica, Enterobacter aerogenes, Enterobacter cloacae, Enterococcus faecalis, Escherichia coli, Escherichia hirae, Flavobacterium meningosepticum, Fluoribacter bozemanae, Francisella tularensis, Francisella tularensis biovar Tularensis, Francisella tularensis subsp. Holarctica, Francisella tularensis subsp. nearctica, Francisella tularensis subsp. Tularensis, Francisella tularensis var. palaearctica, Fudobascterium nucleatum, Fusobacterium necrophorum, Haemophilus ducreyi, Haemophilus influenzae, Helicobacter pylori, Kingella kingae, Klebsiella mobilis, Klebsiella oxytoca, Klebsiella pneumoniae, Lactobacillus acidophilus, Lactobacillus casei, Lactobacillus hilgardii, Lactobacillus pentosus, Lactobacillus plantarum, Lactobacillus rhamnosus, Lactococcus lactis, Legionella bozemanae corrig., Legionella pneumophila, Leptospira alexanderi, Leptospira borgpetersenii, Leptospira fainei, Leptospira inadai, Leptospira interrogans, Leptospira kirschneri, Leptospira noguchii, Leptospira santarosai, Leptospira weilii, Leuconostoc lactis, Leuconostoc oenos, Listeria ivanovii, Listeria monocytogenes, Moraxella catarrhalis, Morganella morganii, Mycobacterium africanum, Mycobacterium avium, Mycobacterium avium subspecies paratuberculosis, Mycobacterium bovis, Mycobacterium bovis strain BCG, Mycobacterium intracellulare, Mycobacterium kansasii, Mycobacterium leprae, Mycobacterium marinum, Mycobacterium tuberculosis, Mycobacterium typhimurium, Mycobacterium ulcerans, Mycoplasma hominis, Mycoplasma mycoides, Mycoplasma pneumoniae, Neisseria gonorrhoeae, Neisseria meningitidis, Neorickettsia sennetsu, Nocardia asteroides, Orientia tsutsugamushi, Pasteurella haemolytica, Pasteurella multocida, Plesiomonas shigelloides, Propionibacterium acnes, Proteus mirabilis, Proteus morganii, Proteus penneri, Proteus rettgeri, Proteus vulgaris, Providencia alcalifaciens, Providencia rettgeri, Pseudomonas aeruginosa, Pseudomonas mallei, Pseudomonas pseudomallei, Pyrococcus abyssi, Rickettsia akari, Rickettsia canadensis, Rickettsia canadensis corrig, Rickettsia conorii, Rickettsia montanensis, Rickettsia montanensis corrig, Rickettsia prowazekii, Rickettsia rickettsii, Rickettsia sennetsu, Rickettsia tsutsugamushi, Rickettsia typhi, Rochalimaea quintana, Salmonella arizonae, Salmonella choleraesuis subsp. arizonae, Salmonella enterica subsp. Arizonae, Salmonella enteritidis, Salmonella paratyphi, Salmonella typhi, Salmonella typhimurium, Selenomonas nominantium, Selenomonas ruminatium, Serratia marcescens, Shigella boydii, Shigella dysenteriae, Shigella flexneri, Shigella sonnei, Spirillum minus, Staphylococcus aureus, Staphylococcus epidermidis, Staphylococcus equi, Staphylococcus lugdunensis, Stenotrophomonas maltophila, Streptobacillus moniliformis, Streptococcus agalactiae, Streptococcus bovis, Streptococcus ferus, Streptococcus pneumoniae, Streptococcus pyogenes, Streptococcus viridans, Streptomyces ghanaenis, Streptomyces hygroscopicus, Streptomyces phaechromogenes, Treponema carateum, Treponema denticola, Treponema pallidum, Treponema pertenue, Vibrio cholerae, Vibrio parahaemolyticus, Vibrio vulnificus, Xanthomonas maltophilia, Yersinia enterocolitica, Yersinia pestis, Yersinia pseudotuberculosis, and Zymomonas mobilis.

In further preferred examples, pathogenic bacteria harbouring a CRISPR-Cas system are selected from the group consisting of: Bacillus anthracis, Campylobacter jejuni, Clostridium botulinum, Clostridium difficile, Corynebacterium diphtheria, Corynebacterium pseudotuberculosis, Escherichia coli, Enterococcus faecalis, Erwinia amylovora, Francisella cf novicida, Gardnerella vaginalis, Helicobacter pylori, Klebsiella pneumoniae, Legionella pneumophila, Leptospira interrogans, Listeria monocytogenes, Mannheimia haemolytica, Mycobacterium bovis, Mycobacterium tuberculosis, Mycoplasma gallisepticum, Mycoplasma pneumoniae, Neisseria meningitides, Pseudomonas aeruginosa, Salmonella enterica, Shigella flexneri, Staphylococcus epidermidis, Staphylococcus aureus, Streptococcus pyogenes, Vibrio cholera, Yersinia pseudotuberculosis, Yersinia pestis, as referenced in whole or in part by Grissa et al., BMC Bioinformatics. vol. 8, pp 172 (2007); Makarova et al., Nat. Rev. Microbiol., vol. 13 pp 722 (2015).

As used herein, an “autoinducer” or “AI” (or collectively referred to as “autoinducers” or “AIs”) is defined as a molecule that activates the expression of QS-regulated genes. In some embodiments, the autoinducer is produced by the microorganism, while in other embodiments, the autoinducer is produced synthetically or obtained from microorganisms and added to mutant strains (e.g., QS deficient strains of bacteria) growing in media.

As used herein, “biofilms” are defined as sessile microorganism communities, such as bacterial and/or fungal communities, that occupy a surface. Biofilms are surface-associated assemblies of microorganisms, such as bacteria and/or archaea which are bound together by extracellular polymeric substances (4, 5). Biofilms are attached to the surface all along the edges, including the bottom edge, of the surface. Although bacterial biofilms are desirable in waste-water treatment (6), biofilms primarily cause undesirable effects such as chronic infections in patients; medical device-associated infections, clogging, and/or device failure; as well as clogging of infrastructure of pharmaceutical manufacturing equipment and other industrial fluid flow systems (1-3). Biofilms also provide a barrier for bacterial communities, which often hinders or impedes treatment with antibiotics. For example, cells in biofilms display many behavioral differences from planktonic cells, such as a 1,000-fold increase in tolerance to antibiotics (7, 8), an altered transcriptome (9-11), and spatially heterogeneous metabolic activity (12, 13). Some of these physiological peculiarities of biofilm-dwelling cells may be due to strong gradients of nutrients and metabolites, which also affect biofilm morphology and composition (14, 15).

As used herein, “biofilm streamers” are defined as biofilms that have been partially detached from the surface upon which the biofilm is growing. Under conditions of fluid flow in the presence of available biofilm promotion element(s) (e.g., curves, corners, bends, etc.), the flow partially detaches the extracellular matrix from the substrate along with cells embedded in the extracellular matrix and the film is suspended in the liquid, attached only at its edges. The detached biofilm forms filaments or streamers in the flowing liquid. The streamer is then able to capture other flowing debris and cells in order to continue growing. Thus, biofilms grow by cellular division, while biofilm streamers grow both by cell division as well as cellular capture of passing cells in the flow.

As used herein, “biofilm growth” is defined as the expansion of the surface-attached biofilm over time, whether through cell division or through attachment of additional cells to the surface from the surrounding environment. As used herein, this growth includes expansion laterally over available surfaces as well as expansion into the third dimension through thickening of the biofilm layer by additional layers of cells.

As used herein, “biofilm morphology” is defined as the physical composition or shape of the biofilm. As used in the invention, biofilm morphology may change over time. These changes may be in composition of the extracellular matrix, in the composition of microorganisms, such as bacteria and/or archaea in the biofilm, or in the shape of the biofilm. Biofilm growth would be an example of a change in biofilm morphology. Another example of a change in biofilm morphology would be the flow induced formation of biofilm streamers. A third example would be the inclusion or expulsion of different microbial species within the biofilm.

As used herein, “biofilm streamer growth” is defined as the expansion of the biofilm streamer over time. As used herein, this expansion may be in the length of the biofilm streamer filaments and/or in the thickness of the biofilm streamer. This growth may be through cell division and/or through capture of additional cells, extracellular matrix, and/or debris from the surrounding liquid.

As used herein, “biofilm streamer morphology” is defined as the physical composition and/or shape of the biofilm streamer. As used in the invention, biofilm streamer morphology may change over time. These changes may be in the extracellular matrix, in the composition of the microorganisms (e.g., bacteria and/or archaea) in the biofilm streamer and/or in the shape of the biofilm streamer. Biofilm streamer growth, fluid flow induced formation and/or inclusion/exclusion of different microbial species are all examples of a change in biofilm streamer morphology.

As used herein, “QS inhibitors” or QS antagonists are defined as any molecule that inhibits QS, QS controlled gene expression, a biofilm (or formation thereof), a biofilm streamer (or formation thereof), and/or a virulence factor production. QS inhibitors may be used to treat infections (including bacterial infections and bacterial infections resistant to antibiotics). In some embodiments, QS antagonists include, but are not limited to, small organic molecules, peptides, and synthetic molecules.

As used herein, “QS agonists” or “agonists” are defined as any molecule that activate QS or QS controlled gene expression to repress a biofilm (or formation thereof), a biofilm streamer (or formation thereof), and/or a virulence factor production. QS agonists may be used to treat infections (including bacterial infections and bacterial infections resistant to antibiotics such as cholera). In some embodiments, QS agonists include, but are not limited to, small organic molecules, peptides, and synthetic molecules.

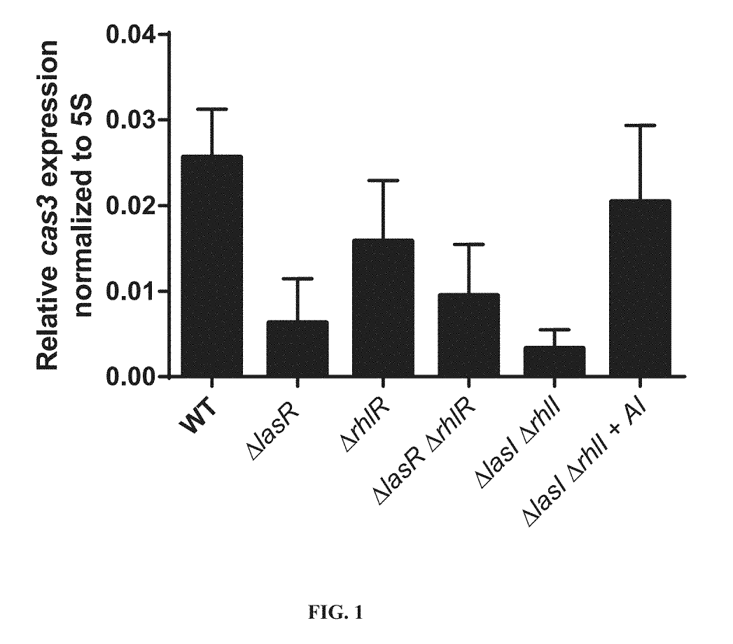

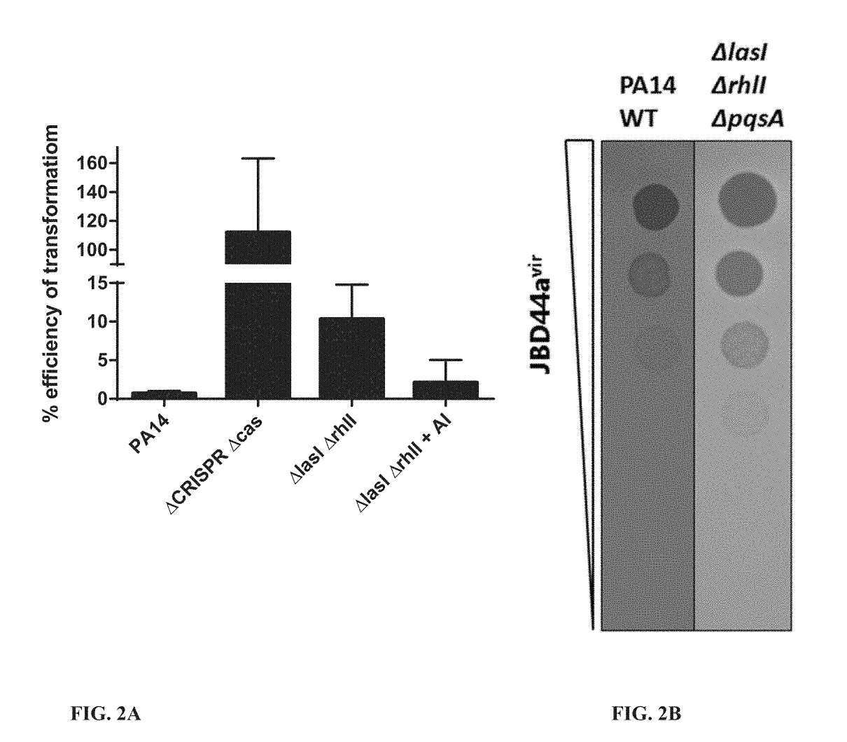

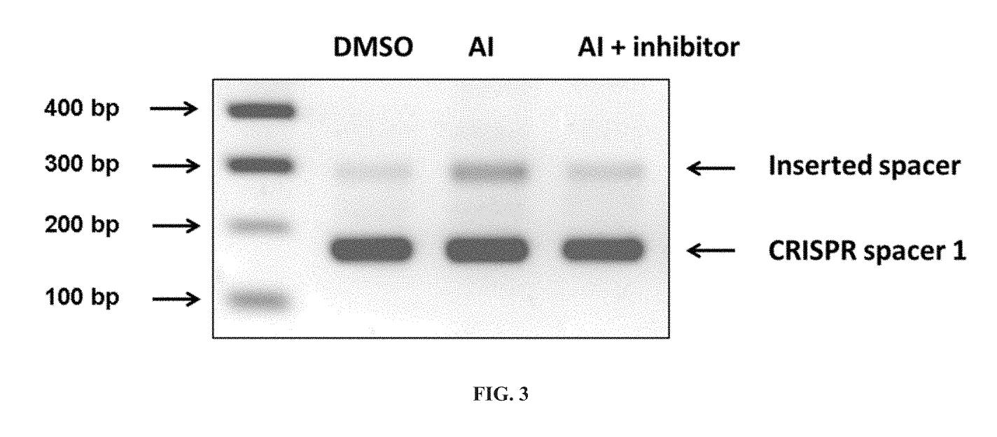

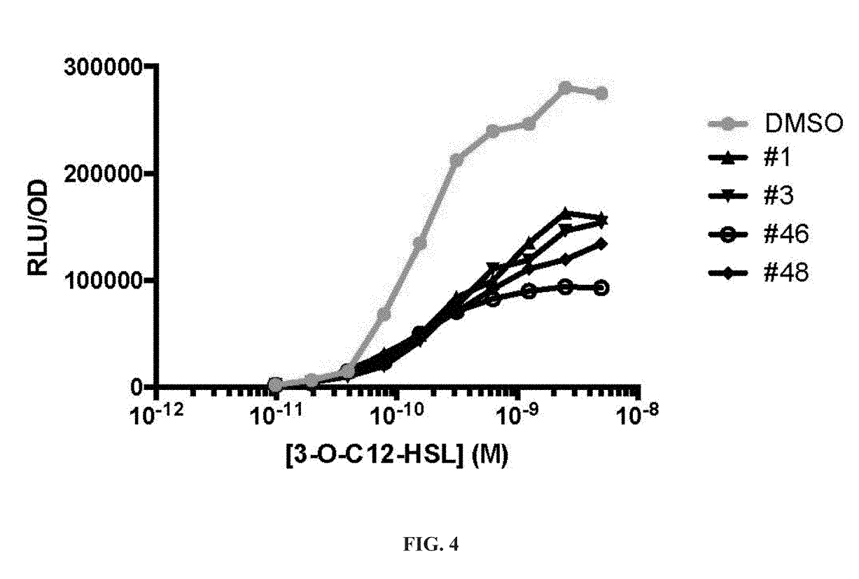

Examples of QS inhibitors/antagonists are described in U.S. Pat. Nos. 8,247,443, 8,568,756, or PCT/US14/56497, and are specifically incorporated by reference in their entirety. See, for example, the structures described in FIGS. 2, 8 and 9 of U.S. Pat. No. 8,247,443, FIGS. 3A-P, 4A, 8A-8L and 10A-B of U.S. Pat. No. 8,568,756, and in Tables 1-4 and FIGS. 1, 6, 7, 12-15 of PCT/US14/56497, all of which are herein incorporated by reference in their entirety.

Examples of QS agonists are described in U.S. Pat. No. 8,535,689 or WO 2014/092751, and are specifically incorporated by reference in their entirety.

As used herein, “lytic phage” is a bacterial virus (bacteriophage) that infects bacteria or archaea and causes lysis and destruction of the bacterial or microorganism cell after replication of the phage. As soon as the cell is destroyed, the phage progeny can find new host cells (e.g., bacteria) to infect. In general, lytic phages are more suitable for phage therapy than lysogenic phages as the lysogenic cycle does not lyse the host cell. In preferred embodiments, lytic phages do not comprise toxins or other materials that would have an adverse impact on the human, animal or plant recipient. Unless otherwise indicated, “phage” or “phage” or “lytic phage” refers to a lytic phage.

As used herein, phages include both naturally occurring and engineered phages (e.g., phages that are not naturally occurring). Engineered phages may include, but are not limited to, lytic phages that have been optimized for specific properties through screening techniques known in the art, lysogenic phages that have been genetically engineered to express lytic proteins and therefore have become lytic, etc.

As used herein, a “phage cocktail” is defined as a mixture of one or more different types of phage that can infect the same strain or different strains of bacteria. Phage cocktails can be formulated as compositions, such as but not limited to, pharmaceutical compositions.

As used herein, “phage therapy” is defined as the therapeutic use of phages to treat infections from microorganisms (e.g., bacterial infections, especially pathogenic and antibiotic resistant bacterial infections). In some embodiments, phages are specific to a particular species of bacteria. Examples of phages used in phage therapies are described in Table 4, and the corresponding references of Table 4 are specifically incorporated by reference herein in their entirety.

Unless otherwise indicated, “host” refers to a bacterial host. Unless otherwise indicated, “recipient” refers to a human, animal, plant, etc. that receives the QS inhibitors/agonists and/or phage therapies described herein.

As used herein, the term “composition” or “pharmaceutical composition” may include one or more of a QS inhibitor/agonist, a combination of one or more QS inhibitors/agonist and one or more phages (preferably lytic phages) used in phage therapy, one or more antibiotics, or any other material used to treat or prevent infections.

As used herein, the surface (e.g., surface of a medical device, of an industrial pipe, etc.) can be made of any material. For example, glass, metals, including, but not limited to stainless metals, silicon, plastic, polymers, metals, and/or ceramic materials can be used. Surfaces also include human tissues or skin.

Preferred examples of surfaces that can be treated with the compositions of the present invention include, but are not limited to a surface comprising polymers, such as, for example, polyethylene, polypropylene, polystyrene, polyester, polyester PLA and other bioabsorbable plastics, polycarbonate, polyvinyl chloride, polyethersulfone, polyacrylate (e.g., Acrylic, PMMA), hydrogel (e.g., acrylate), polysulfone, polyetheretherketone, thermoplastic elastomers (e.g., TPE, TPU), thermoset elastomers, silicone, poly-p-xylylene (e.g., Parylene), fluoropolymers, a metal, including, but not limited to stainless steel, cobalt-base alloys, titanium, titanium-base alloys, and/or shape memory alloy, and/or a ceramic material including, but are not limited to glass ceramics, calcium phosphate ceramics, and/or carbon-based ceramics). Moreover, the surface can have any shape, such as, for example, flat and/or curved surfaces as described herein.

As used herein, the surface may comprise of a “channel” which is defined as a passage directing the flow of a fluid. As used in the invention, a channel may be an enclosed hollow tube. The cross section of the tube may be of any suitable geometry as is known by those of skill in the art. In one example the cross section is circular, oval, square, rectangular and/or irregularly shaped. The tube may have a constant cross-sectional area and/or it may be variable (e.g. it may constrict in certain areas and/or expand in others). The cross section of the channel may change shape along its length. In other examples, the channel may be a depression, gutter, groove and/or furrow. This depression may be shallow, deep, narrow and/or wide. In other examples, the channel may be provided by the gap between two parallel flat planar surfaces placed close together. In still other examples, the channel may be part of a larger device or machine or biological tissue or organ (e.g., lungs). The channel may be a fluid flow conduit in an implantable medical device. The channel may also be a fluid flow conduit in machinery used in industrial processes. The channel may be very small (i.e. just large enough for fluid and bacterial or fungal cells to flow through) or very large (i.e. the large culverts and pools used in a waste water treatment facility.)

As used herein, “edge” is defined as a line or line segment that is the intersection of two plane faces.

As used herein, an “object” comprises a surface. In some examples, an object may include, for example, sand, gravel, granules and the like. In other examples, an object may include portions of medical devices or industrial fluid handling machinery. For example, an object may include filter support grids, filter mesh, stents, tubing or channel components for fluid handling, valves, pumps, and the like. These objects may be of any scale from miniature components of implantable medical devices to large scale fluid handling components of industrial cooling units or food processing machinery. Objects also include human tissues, organs, or skin.

As used herein, “stent” is defined as a mesh tube inserted into a natural passage/conduit in the body to prevent localized flow constriction, including bare-metal stents.

As used herein, “bare-metal stent” is defined as a type of vascular stent without a coating (as used in drug-eluting stents, for example). Stents are made out of different types of fabrics, polymers, and other materials, such as for example, bare stainless steel or may be made of alloys (e.g., cobalt chromium).

As used herein, “pipe” is defined as a generally rigid tube used to convey fluid or compressed gases. A pipe is made of glass, any number of metals, any number of plastics or other polymeric materials, or concrete. A pipe as used herein may be any that is known to one of ordinary skill in the art.

As used herein, “fluid” is defined as a liquid or a gas. In one example, the fluid is water, with or without the addition of other components. These additional components may include, but are not limited to nutrients and salts needed to support bacterial or microorganism growth, and may include chemical or biochemical probes to assist with visualization of cells or extracellular components, test compounds, and compounds for selective growth of specific bacterial strains. In other embodiments, fluid may include media or other materials used in large or small scale manufacturing processes. In other embodiments, a fluid is a biological fluid such as, for example, blood.

As used herein, “fluid flow” is defined as movement of the fluid along a surface, e.g., within a pipe, a channel or equivalent in a continuous stream.

As used herein, “fluid flow rate” is defined as the volume of a fluid moving along a surface, e.g., within a pipe, a channel or equivalent per unit time.

As used herein, “test compound” is defined as any compound added to the test system for evaluation of its effect on QS, biofilm formation, biofilm streamer formation, virulence factor production. The effect of the test compound may be to inhibit biofilm, biofilm streamer, and/or virulence factor production as well as to treat infections (including bacterial infections and bacterial infections resistant to antibiotics). The inhibition of QS, biofilm formation, biofilm streamer formation, virulence factor production, and/or infections (including bacterial infections and bacterial infections resistant to antibiotics) through the use of a QS antagonist preferably leads to a decrease in overall virulence and infectivity to the recipient. In other embodiments, the test compound may agonize QS receptors to ultimately inhibit pathogenicity and biofilm formation.

These compounds (e.g., QS inhibitors/agonists) may be pharmaceutical compounds, such as small molecules.

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

### B. Uses of OS Compounds and OS Compounds with Phage Therapy

The compositions described herein can be used to inhibit/agonize QS to repress biofilm formation, biofilm streamer formation, virulence factor production and/or infections by microorganisms (including bacterial infections and bacterial infections resistant to antibiotics) or any other QS-controlled trait of interest. For example, present invention embodiments can reduce and/or prevent QS, biofilm formation, biofilm streamer formation, and/or virulence factor production on structures or surfaces susceptible to colonization and/or clogging by microorganisms. Compositions can be used in industrial or clinical settings to inhibit or prevent biofilm formation, biofilm streamer formation, virulence factor production to treat infections from microorganisms, including removal of antibiotic resistant bacteria from e.g., hospitals or other public settings.

In some embodiments, the QS inhibitors are flavonoids (e.g., quercetin, baicalein) that inhibit LasR and RhlR, the master regulators of QS pathways in P. aeruginosa, and inhibit virulence factor production and block the formation of normal biofilms. In P. aeruginosa, QS (as shown herein) regulates CRISPR-Cas, a bacterial “adaptive immune system” that activates the bacterium's ability to eliminate phage infections. By blocking QS with a flavonoid or other suitable QS inhibitor, one can suppress the bacterial immune system and enable the use of phage therapy to efficiently target and kill immunocompromised P. aeruginosa. Since QS regulates adaptation (via the CRISPR-Cas system, as shown herein), a process by which bacterial cells acquire immunity against phages, bacterial adaptation to phage therapy can be minimized or prevented by inhibiting QS.

These compositions have immediate applications for medical and health-care devices in which pathogenic microorganisms, such as for example, S. aureus, colonizes. The compositions as described herein inhibit biofilm formation, biofilm streamer formation and toxin synthesis of a microorganism by interfering with the QS regulatory network, thereby reducing the severity of infection and/or colonization of microorganisms including bacteria within or on patients, within or on medical devices, or within or on other surfaces susceptible to microorganisms including bacterial colonization. Furthermore, the compositions are suitable for treatment of bacterial strains that are antibiotic-resistant, and do not promote bacterial resistance, leading to improved treatments for bacterial infections. The compositions can be applied to other devices or settings in which bacterial or microorganism contamination is a concern. Examples include water supply lines, and indwelling medical devices such as filters, stents, intubation tubes, etc. In addition, the compositions are suitable for treatment of a broad range of other Gram-positive and/or Gram-negative pathogens that use QS pathways to control virulence, including but not limited to, Staphylococcus epidermidis, Streptococcus pneumonia, Streptococcus mutans, Streptococcus sanguinis, and P. aeruginosa.

In some embodiments, the compositions described herein can be used to wash, rinse or swab floors and counters or other surfaces prone to microbial/bacterial contamination, such as in food preparation areas, medical facilities, or manufacturing facilities. In other embodiments, the compositions can be used to remove biofilms that have grown on hospital surfaces, and in moist and warm environments, such as showers, water and sewage pipes, cooling or heating water systems, in water supplies, etc. In still other embodiments, the compositions described herein may be used to treat a pool used in a waste water treatment facility, or equipment used in a waste water treatment facility.

In other embodiments, compositions may be used to treat or disinfect pipes, industrial machinery used in manufacturing processes, fluid handling machinery and other equipment associated with pharmaceutical production, agricultural processing, food manufacturing, as well as treatment of food supplies, and/or other machinery. In yet other embodiments, the compositions may be used to treat industrial fluid handling systems or other systems where fluid is directed along channels. For example, the compositions may be used to treat component parts or particular materials in microfluidic or other benchtop-sized assay systems.

The compositions described herein can be used to coat or disinfect implantable medical devices (e.g., stents, catheters, intubation tubes, ventilator equipment, etc.), a wound on the surface or within the body of a patient, as part of a medical or surgical process, and for sterilization of surgical equipment. Still further, these compositions can be added to a handwash to help eliminate spread of virulent bacteria and microorganisms by health workers, patients and others.

Particular species of bacteria may be especially problematic. For example, Pseudomonas aeruginosa is a pathogen that can survive in a wide range of environments. This bacterium is a public health threat because it causes a variety of secondary infections in humans, in which those with burn wounds, cystic fibrosis, and implanted medical devices, or cancer patients receiving chemotherapy are particularly at risk. With an outer membrane of low permeability, a multitude of efflux pumps, and various degradative enzymes to disable antibiotics, P. aeruginosa is difficult to treat. As with other common pathogenic bacteria, antibiotic-resistant strains of P. aeruginosa are an increasing problem.

Biofilm formation in bacterial species, e.g., P. aeruginosa, is often a barrier to phage infection in biologically relevant systems. QS, which has been implicated in activating biofilm formation, is a novel therapeutic target for developing new therapies to inhibit bacterial infection. QS inhibitors, e.g., small molecule compounds, may be used to (1) inhibit biofilm formation in P. aeruginosa in a manner that is essentially equivalent to a phenotypic loss of function of the LasR and/or RhlR QS receptor.

Thus, the compositions described herein (e.g., a QS inhibitor or a QS inhibitor in combination with a phage therapy, and optionally, in combination with one or more other substances that may be used to treat infections) can be used to treat infections (such as for example, sepsis or respiratory infections in patients with cystic fibrosis) in a patient, particularly those patients having infections caused by antibiotic resistant strains of bacteria. In preferred embodiments, the bacterial infection is caused by any pathogenic bacteria capable of QS and having a CRISPR-Cas system, e.g., P. aeruginosa and/or S. aureus.

In other embodiments, as described herein, QS agonists may be used to treat cholera infections or contamination of water supplies by cholera.

Blocking virulence is one of the strategies contemplated to combat these microorganisms/bacteria. This approach, specifically use of QS inhibitors/agonists, provides less selective pressure for the spread of resistant mutants and leads to drug therapies that are effective over a greater time/life span as compared to traditional antibiotics or other therapies. Rather than preventing growth or killing the microorganisms/bacteria, an antivirulence approach prevents the expression of virulence traits. The microorganisms/bacteria that have been treated are thus benign and can then be more easily cleared by the recipient's immune system.

In a further embodiment, methods of screening for antagonists of QS, biofilm formation, biofilm streamer formation, virulence factor production and/or inhibition or treatment of infections (including bacterial infections and bacterial infections resistant to antibiotics) can be performed. These screens may additionally be run in the presence of various phage therapies to determine effective combinations of QS inhibitors and phages for treatment of antibiotic resistant bacteria. Further, these methods of screening may be run in the presence of one or more antibiotics or other substances to detect compositions that enhance antibiotic inhibition of bacteria.

In another embodiment, methods of screening for agonists of QS to inhibit biofilm formation, biofilm streamer formation, virulence factor production and/or inhibit or treat of infections (including bacterial infections and bacterial infections resistant to antibiotics such as cholera) can be performed. These screens may additionally be run in the presence of various phage therapies to determine effective combinations of QS agonists and phages for treatment of antibiotic resistant bacteria. Further, these methods of screening may be run in the presence of one or more antibiotics or other substances to detect compositions that enhance antibiotic inhibition of bacteria.

### C. CRISPR-Cas

It is known that phages viruses have the capacity to invade and destroy bacteria and other microorganisms. Phage replicate and spread when they encounter susceptible hosts. For example, bacteria at high cell densities are particularly at high risk for phage infection, as this situation provides optimal conditions for phage proliferation.

Bacteria, in order to promote their own survival, have evolved multiple phage defense mechanisms to evade attack and prevent their destruction by the spread of phage progeny. One such mechanism is the CRISPR-Cas (clustered regularly interspaced short palindromic repeats with its −CRISPR-associated proteins) system, which is an adaptive immune system that bacteria utilize to fight infection by phage. The CRISPR-Cas system detects and cleaves foreign genetic material, such as phage and/or plasmid DNA comprising a CRISPR-targeted sequence, and integrates small fragments of this foreign DNA into its own bacterial genome (e.g., at the CRISPR locus) in a process referred to as adaptation. By expressing the foreign genetic material inserted in the CRISPR locus, mature CRISPR RNAs are generated, that in complex with Cas proteins, function to target and eliminate foreign DNA based on sequence complementarity. Accordingly, a heritable genetic memory of prior infections is generated in the bacterial genome that provides resistance to future infections. Samson et al. “Revenge of the phages: defeating bacterial defences” Nat Rev Microbiol, vol. 11, pp 675-687 (2013) provides additional information about such processes.

According to present invention embodiments, methods of using a monotherapy (e.g., using novel compounds) to inhibit QS activation pathways and thereby inhibit QS, biofilm formation, biofilm streamer formation, virulence factor production, and/or infections (including bacterial infections and bacterial infections resistant to antibiotics) are provided. Small molecule inhibitors of the QS activation pathway in P. aeruginosa are provided herein.

According to other embodiments of the invention, methods of using a combination therapy (e.g., one or more novel compounds in combination with one or more types of phage therapy and optionally one or more antibiotics or other antimicrobial substances) to inhibit QS activation pathways, and thereby inhibit QS, biofilm formation, biofilm streamer formation, virulence factor production, and/or infections (including bacterial infections and bacterial infections resistant to antibiotics) are provided. Since QS regulates both biofilm formation, a major mechanism of antibiotic resistance, and CRISPR-Cas mediated immunity (as shown herein), a mechanism of phage resistance, disabling QS would sensitize the bacteria to phage killing and would additionally prevent the bacteria from gaining adaptive immunity to the phage. For example, the human pathogen P. aeruginosa uses QS at high cell density to activate expression of CRISPR-Cas genes encoding its adaptive immune system. By disabling the CRISPR-Cas system in P. aeruginosa via pharmacological inhibition of QS (as shown herein), the pathogen's susceptibility to phage-mediated killing is increased. Accordingly, combination therapies including one or more novel compounds that inhibit QS and one or more phage therapies are provided. As an example, a QS inhibitor in combination with a P. aeruginosa-specific phage may be used to treat P. aeruginosa infection. The combination QS-inhibitor/phage antimicrobial strategy can be generalized to any pathogenic bacterium that uses QS to control CRISPR-Cas.

Present invention embodiments also include activation of QS (in some systems such as cholera) to inhibit biofilm formation, biofilm streamer formation, virulence factor production, and/or infections (including bacterial infections and bacterial infections resistant to antibiotics) by: (a) a monotherapy using novel compounds, such as small molecule agonists, to activate QS pathways; and/or (b) a combination therapy including novel compounds to activate QS along with phage therapy (and optionally, in combination with other antimicrobials).

This combination therapy has major implications for the treatment of all bacterial infections resistant to antibiotics, including drug resistant P. aeruginosa, and can also be applied to non-clinical antimicrobial products such as hospital disinfectants, water and sewage treatment, and animal husbandry, where phages are already being safely used in agriculture (e.g., applied to plants, used during food production or harvesting, and so forth).

The P. aeruginosa QS circuit comprises two AI synthase/receptor pairs, LasI/R and RhlI/R, which produce and detect 3-O—C₁₂-HSL and C₄—HSL, respectively. At high cell density, LasR and RhlR bind their cognate ligands, dimerize, bind to DNA, and activate expression of genes encoding functions required for virulence and biofilm formation. This QS regulatory network is responsible for controlling the expression of approximately 10% of P. aeruginosa genes.

### D. Phage Therapy

Phage therapy is the therapeutic use of phages, a type of virus, to treat pathogenic bacterial infections. Phages are highly specific, thereby minimizing undesirable side effects due to non-specificity and are generally non-toxic to the recipient. Some phages also have the ability to penetrate biofilms, which act as barriers to traditional antibiotic therapies. Phages may infect bacteria and other microorganisms such as archaea.

Phages may be classified into two general groups: lytic phages, which invade bacterial cells, disrupt bacterial metabolism, and ultimately result in lysis of the bacterial cell, and temperate phages, which integrate their genome into the chromosome of the host bacteria. Present invention embodiments are directed towards lytic phages, which result in lysis and destruction of the pathogenic bacterial or microorganism.

Phages may need infrequent administration, as phages may replicate at the site of infection. In some embodiments, phages continue to replicate at the site of infection as long as their target, e.g., a pathogenic bacterium, is present. For example, administration of a single dose of phage may be present in the bloodstream of the recipient within several hours and in internal organs within about 12 hours. Lytic phages introduce their viral DNA into the bacterial host cell, produce progeny phage, and the bacterial host lyses in the process, thereby killing the pathogenic cell. Then, the phage proceeds to infect surrounding pathogenic bacteria. In some embodiments, phage may remain in the human body for one day, two days, three days, four days, or up to a week. Typically, once the bacterial infection has been eradicated, the phage is eliminated from the recipient, without leaving residual virus behind.

In addition, bacteria that become susceptible to one phage are generally still susceptible to other phages. Additionally, phages are easily administered, e.g., nasal, oral, aerosol, immersion, injection, in food products, and/or topically.

Moreover, phage provide an effective mechanism for targeting specific disease-causing agents (e.g., pathogenic bacteria) without eliminating beneficial bacteria.

As phages are generally quite specific, infecting only targeted bacterial species, prior to initiating phage therapy, it is sometimes desirable to identify the species of bacteria responsible for the infection to ensure that a suitable phage is selected for treatment.

In some embodiments, vectors/plasmids (including a region for antibiotic resistance) or any equivalent comprising a foreign fragment of DNA are used to mimic infection with phages. In such systems, if the CRISPR-Cas system of the bacterial cell successfully integrates the foreign fragment of DNA into its genome, the bacterium gains sensitivity to antibiotics. On the other hand, if the plasmid is able to replicate, antibiotic resistance will be conferred to the bacterial cell, and these cells may be identified by plating on a selective medium and screening for colonies. Construction of various types of vectors/plasmids to mimic the CRISPR-Cas system are described herein, see, e.g., Example 2.

In general, polynucleotides, e.g., polynucleotides encoding a foreign fragment of DNA, targeted by the CRISPR-Cas system, can be incorporated into any desired DNA or RNA based vector, without limitation. For example, a polynucleotide may be cloned into an expression vector, a subcloning vector, a shuttle vector, a vector designed for use with in vitro transcription reactions, cosmids, phagemids, and vectors derived from mammalian viruses, including retroviruses (for example, lentiviruses), adenoviruses, adenoassociated viruses (AAV), and episomal EBNA-based vectors of Epstein-Barr virus origin. In some embodiments, vectors may be in circular form or in linearized form.

One of skill in the art will understand that a wide variety of expression vectors/plasmids are within the scope of present invention embodiments. An expression vector can be optimally designed to express a protein in a host cell. For example, a vector may comprise a nucleotide sequence encoding a protein (e.g., an open reading frame (ORF)), and suitable regulatory elements can be delivered into the host cell by any suitable method of transfection, transduction, etc. Any type and any quantity of regulatory elements, involved in regulation of transcription or translation may be incorporated into the expression vector and may be located upstream or downstream of the ORF. Once in the host cell, the host cell's own machinery, e.g., endogenous RNA polymerases, etc., may be employed to synthesize mRNA, which is translated to produce the protein. In other embodiments, expression vectors are optimally designed to express a functional RNA molecule.

In some embodiments, the expression vectors may be bacterial expression vectors, e.g., such as pCS26 and pBAD-A, and may contain one or more promoter elements, e.g., a phage promoter element such as an araBAD promoter.

### E. Compositions

Present invention embodiments include a novel approach to inhibit QS, biofilm formation, biofilm streamer formation, virulence factor production, and/or infections (including bacterial infections and bacterial infections resistant to antibiotics) by: (a) a monotherapy using novel compounds, such as small molecule inhibitors, to inhibit QS activation pathways; and/or (b) a combination therapy including novel compounds to inhibit QS along with phage therapy (and optionally, in combination with other antimicrobials). Administration of QS inhibitors (QS antagonists), or a combination of QS inhibitors and phage therapy, results in significant reductions in biofilm formation and virulence factor production of microbial organisms as well as inhibition of infections (including bacterial infections and bacterial infections resistant to antibiotics).

Present invention embodiments relate to a method of inhibiting QS, biofilm production, biofilm streamer production, virulence factor production and/or infections (including bacterial infections and bacterial infections resistant to antibiotics) by a microorganism using (1) a small molecule antagonist to decrease or inhibit QS, biofilm production, biofilm streamer production, virulence factor production and/or infections (including bacterial infections and bacterial infections resistant to antibiotics) by a microorganism or (2) a combination therapy of a small molecule antagonist and phage therapy (and optionally, in combination with other antimicrobials) to decrease or inhibit QS, biofilm production, biofilm streamer production, virulence factor production and/or infections (including bacterial infections and bacterial infections resistant to antibiotics) by a microorganism. Experimental evidence is provided herein, showing that specifically targeting receptors in QS pathways inhibits bacterial adaptivity mediated by the CRISPR-Cas system.

It is understood that in some systems (such as cholera), activation of QS represses biofilm formation and pathogenicity. Accordingly, present invention embodiments also include activation of QS to inhibit biofilm formation, biofilm streamer formation, virulence factor production, and/or infections (including bacterial infections and bacterial infections resistant to antibiotics) by: (a) a monotherapy using novel compounds, such as small molecule agonists, to activate QS pathways; and/or (b) a combination therapy including novel compounds to activate QS along with phage therapy (and optionally, in combination with other antimicrobials).

These techniques broadly apply to any lytic phage and any microorganism capable of QS. Additionally, present invention embodiments are not limited to one type of phage, as multiple types of phages (“phage cocktails”) may be administered in combination with a QS inhibitor/agonist.

### F. OS Inhibitors

Flavonoids are natural products with a wide array of pharmacological effects. A set of structurally related flavonoids exhibiting anti-microbial activities in P. aeruginosa has been discovered along with the corresponding mechanism of QS inhibition (as shown herein). These flavonoids bind to the QS-receptors, LasR and RhlR, and significantly reduce the expression of genes encoding functions required for virulence, biofilm formation, and CRISPR-Cas immunity. These molecules have also been shown to inhibit the QS-receptors directly through prevention of DNA binding. This discovery connects a wealth of anti-microbial pharmacological data with direct QS-inhibition, indicating that targeting QS is a viable therapeutic approach to attacking P. aeruginosa and other types of infections.

While the molecules shown in Tables 1 and 2 were known in the art, the activity of these molecules to inhibit cas expression was unexpected and not previously known in the art. Additionally, it was also not previously known that CRISPR-Cas mediated bacterial immunity is regulated by QS, and that by disabling or inhibiting QS using the compounds of Table 1 or Table 2, P. aeruginosa is sensitized to phage infections, and thereby prevented from gaining adaptive immunity to administered phage.

A generalized chemical structure (Formula I) representative of flavonoid compounds capable of inhibiting QS include:

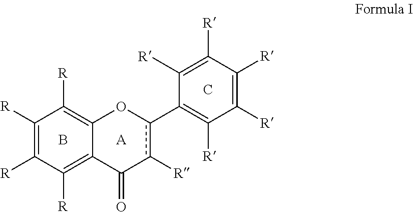

wherein R may be H or OH, and two or more of the R substituents of a given compound are OH; R′ is H or OH; and R″ is H or OH. It was noted that for the A-ring, the presence of the double bond did not appear to be required, as narigenin was also found to be active. A variety of compounds were obtained and tested for activity (e.g., inhibition of receptor specific reporter assays for LasR and RhlR activity). Representative active compounds are shown in Table 1:

TABLE 1

| Compound No. | Chemical Name | Structure |
|---|---|---|
| #3 | chrysin | 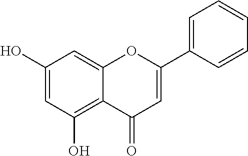 [SMILES: O=c1cc(-c2ccccc2)oc2cc(O)cc(O)c12] |
| #43 | apigenin | 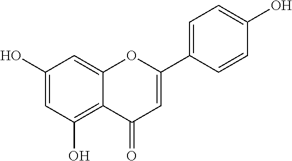 [SMILES: O=c1cc(-c2ccc(O)cc2)oc2cc(O)cc(O)c12] |
| #48 | quercetin | 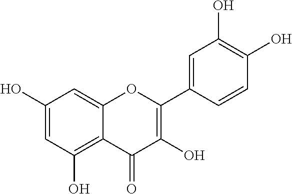 [SMILES: O=c1c(O)c(-c2ccc(O)c(O)c2)oc2cc(O)cc(O)c12] |
| #46 | baicalein | 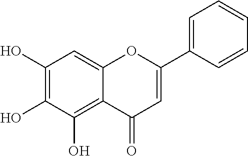 [SMILES: O=c1cc(-c2ccccc2)oc2cc(O)c(O)c(O)c12] |
| #54 | 7,8-dihydroxyflavone | 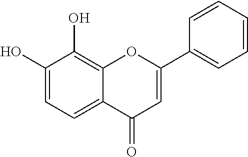 [SMILES: O=c1cc(-c2ccccc2)oc2c(O)c(O)ccc12] |
| #53 | 6-dihydroxyflavone | 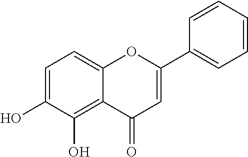 [SMILES: O=c1cc(-c2ccccc2)oc2ccc(O)c(O)c12] |
| #4 | narigenin | 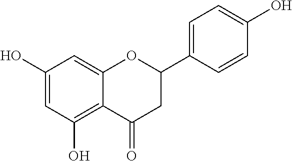 [SMILES: O=C1CC(c2ccc(O)cc2)Oc2cc(O)cc(O)c21] |
| #18 | 3,5,7-trihydroxyflavone | 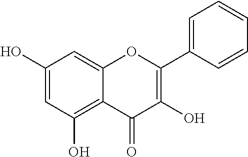 [SMILES: O=c1c(O)c(-c2ccccc2)oc2cc(O)cc(O)c12] |
| #19 | pinocembrin | 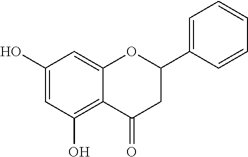 [SMILES: O=C1CC(c2ccccc2)Oc2cc(O)cc(O)c21] |

TABLE 2

| Compound | Chemical | |
|---|---|---|
| No. | Name | Structure |
| #1 | phloretin | 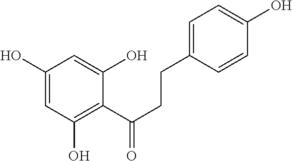 [SMILES: O=C(CCc1ccc(O)cc1)c1c(O)cc(O)cc1O] |

In some embodiments, two (2) or more hydroxyl residues in the R positions (of the B-ring) of the compounds of Formula I are present. Any of these two or more R positions may be hydroxylated.

Table 2 shows another compound, with a related structure to Formula I, also found to be active.

Other representative compounds of present invention embodiments include compounds having general structures as provided in PCT/2014/056497. In one aspect, the invention is a compound having the general structure of Formula II:

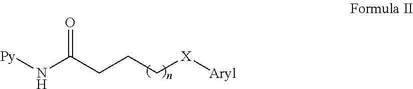

wherein Py is a pyridine ring attached in the 2, 3, or 4 position and substituted with one or more additional substituents selected from the group consisting of alkyl, trifluoromethyl, methoxy, F, Cl and Br; aryl is a benzene ring with one or more additional substituents selected from the group consisting of: methyl, trifluoromethyl, cyano, nitro, F, Cl, Br and methoxy; X is O, NH, S or —CH₂—; and n is 0 to 4 —CH₂— units.

In another embodiment, the compound has the general structure of Formula III:

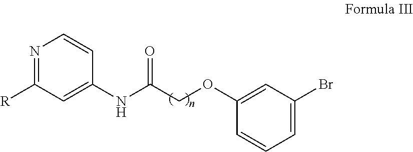

wherein R is a substituent selected from the group consisting of alkyl, trifluoromethyl, methoxy, and Cl; and n is 4 to 5 —CH₂— units.

In yet another embodiment, the compound has the general structure of Formula IV:

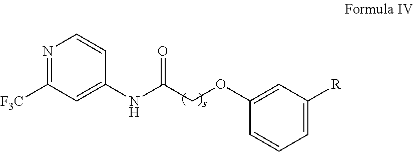

wherein R is a substituent selected from the group consisting of I, F, and Cl.

In another aspect, the invention is a compound having the general structure of Formula V:

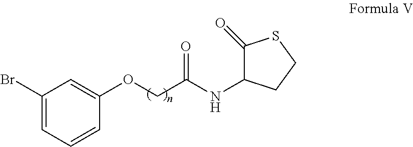

where n=1, 2, 4 or 5.

In yet another embodiment, the compound has the general structure of Formula VI:

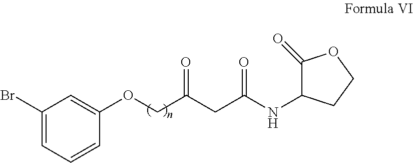

where n=1 or 3.

Other representative compounds of the present invention include compounds having the following general structure, as found in U.S. Pat. No. 8,247,443, 8,568,756, or 8,772,331. For example, in some embodiments, the compound has the general structure of Formula VII:

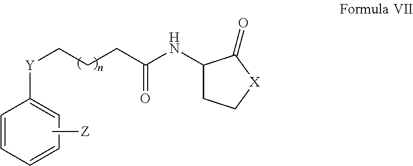

wherein X is O or S (preferably O); n is 0, 1 or 2 (preferably 1); Y is O, S or CH₂ (preferably O); wherein the aryl ring can be substituted (represented by Z) with halogens (preferably chlorine, bromine, or fluorine), hydroxyl, alkoxyl (wherein the alkyl group is preferably methyl, ethyl, propyl, or isopropyl), cyano, nitro, amido, acetamido, amino, alkylamino (wherein the alkyl group is preferably methyl, ethyl, propyl, or isopropyl), aryl, heteroaryl, acyl (wherein the acyl chain is preferably methyl, ethyl, propyl, or isopropyl), alkyl (wherein the alkyl group is preferably methyl, ethyl, propyl, or isopropyl), cycloalkyl (wherein the alkyl group is preferably propyl, butyl, pentyl, or hexyl), sulfonamide, alkyl sulfonamide (wherein the alkyl group is preferably methyl, ethyl, propyl, or isopropyl); wherein the aryl ring substituent Z can occur at the ortho, meta or para position; or wherein the aryl ring can be multiply substituted with the substituents as described above.

Compounds contemplated by present invention embodiments do not include meta-bromothiolactone (mBTL); chlorolactone (CL); and chlorothiolactone (CTL).

In another embodiment, the compounds contemplated according to embodiments of the present invention are represented by the compounds shown in Table 3 as follows:

TABLE 3

| Patent No. | Reference | Compounds |
|---|---|---|
| PCT/US2014/056497 | Table 1 | Entry 1-12 |
| | Table 2 | Entry 1-23 |
| | Table 3 | Entry 1-23 |
| | Table 4 | Entry 1-20 |
| U.S. Pat. No. | FIG. 3 | Antagonist 6807-0002 |
| 8,568,756 | | Antagonist 8008-8157 |
| (same antagonists | | Antagonist C104-0038 |
| also in | | Antagonist C105-2488 |
| U.S. Pat. No. | | Antagonist 3448-8396 |
| 8,772,331 and | | Antagonist 3578-0898 |
| U.S. Pat. No. | | Antagonist 3643-3503 |
| 8,247,443) | | Antagonist 4052-1355 |
| | | Antagonist 4248-0174 |
| | | Antagonist 4401-0054 |
| | | Antagonist 4606-4237 |
| | | Antagonist C137-0541 |
| | | Antagonist C450-0730 |
| | | Antagonist C540-0010 |
| | | Antagonist C646-0078 |
| U.S. Pat. No. | FIGs. 13a-13e | Compounds 1-33, and CAI-1 |
| 8,535,689 | | |
| WO 2014/092751 | FIG. 2A | Compounds 1-11 |
| | FIG. 3 | Compounds 11-18 |

Additional compounds contemplated according to embodiments of the present invention are represented by the compounds shown in Table 3. Unless otherwise indicated, the compounds of PCT/US2014/056497 and U.S. Pat. No. 8,568,756 function as antagonists of QS to inhibit the QS pathway.

It is also expressly understood that the compounds referred to (and incorporated by reference) in PCT/US2014/056497 are limited to those that exhibit anti-pathogenic and anti-biofilm activity through inhibition of QS.

Unless otherwise indicated, the compounds of U.S. Pat. No. 8,535,689 and WO 2014/092751 function as agonists. Some QS systems, such as those found in cholera, have a CRISPR-Cas system that works “in reverse” from other QS systems. For example, agonists of cholera QS receptors repress biofilm formation and pathogenicity, effectively functioning as inhibitors of bacterial infections.

It is expressly understood that present invention embodiments include both agonists and antagonists. In some systems, compounds act as antagonists with respect to the QS system to repress pathogenicity, while in other systems, compounds act as agonists with respect to the QS system to repress pathogenicity.

### G. Phage Therapy

Present invention embodiments include a combination therapy of a QS inhibitor/agonist with phage therapy. Methods for screening phages with specificity to a particular organism or for genetically engineering phages with specific properties are known in the art, see, e.g., WO 00/69269, US 2002/0001590, and US 2002/0044922. Methods for generation of phage compositions for commercial production may be found in U.S. Pat. Nos. 8,178,087 and 7,588,929. Other examples of phage therapy include U.S. Pat. No. 8,282,920. Each of these references are incorporated by reference herein in their entirety.

In some embodiments, phages with specificity to a microorganism may be engineered to have e.g., anti-CRISPR activity and/or anti QS activity. In other embodiments, phages may be genetically engineered to be lytic. In other embodiments, phages having specificity to a particular microorganism may be identified in a screening assay, and may be further optimized using techniques known in the art. In some embodiments, phages target bacterial receptors that are not upregulated by QS so as to not reduce the ability of the phage to infect the host cell.

Embodiments of the present invention include sensitizing bacterial cells to attack by a phage using a QS inhibitor/agonist, and then treating the cell with a lytic phage. Lytic phages that infect a host bacterial cell cause lysis of the bacterial cell at the end of their life cycle, and are thus suitable for the treatment of bacterial infections.

In general, phage strains may be selected from Cystoviridae, Leviviridae, Myoviridae, Podoviridae, Siphoviridae, Corticoviridae, Inoviridae, Microviridae, Lipothrixviridae, Rudiviridae, Ampullaviridae, Bicauddaviridae, Clavaviridae, Fuselloviridae, Globuloviridae, Guttaviridae, Plasmaviridae, and Tectiviridae families, preferably from Myoviridae, Podoviridae and Siphoviridae families, and most preferably in the Myoviridae family. Non-lytic strains could be engineered to become lytic, for use in this application as preferred embodiments of the invention include lytic phages. Lytic phages may be isolated using screening techniques known in the art. In other embodiments, phages may be genetically engineered to be lytic.

The Myoviridae family comprises phages, including phage Mu, P1, P2, and T4, and the “T4-like” genus. The Microviridae family of lytic phages (one preferred embodiment) infects enterobacteria; spiroplasma; bdellovibrio, and chlamidia, and includes G4 and phi×174. The Podoviridae family comprises phages, including phages N4, P22, T3, and T7. The Siphoviridae family includes phages hk022, lambda, T5, BF 23.

Synergy may be observed when administering a combination of a QS inhibitor/agonist and a phage therapy. In some embodiments, a 10%, a 20%, a 30%, a 40%, a 50%, a 60%, a 70%, an 80% a 90% and so forth, synergistic effect may be observed with the combination as compared to treatment with a QS inhibitor/agonist or phage therapy alone.

Table 4 lists examples of phage therapy. This table is intended to be non-limiting. It is expressly contemplated that phages specific to any microorganism listed herein may be obtained using techniques known in the art.

TABLE 4

| Phages used in | | Bacterial |
|---|---|---|
| Phage Therapy | Reference | Organism |
| OVC8 | Solis-Sanchez et al., “Genetic | Vibrio cholerae |
| | characterization of OVC8 lytic | |
| | phage for Vibrio Cholerae O1”, | |
| | Virol J., (2016), vol. 13, pp 47. | |
| KPP21 | Shigehisa et al., “Characterization | Pseudomonas |
| | of Pseudomonas aeruginosa | aeruginosa |
| | phage KPP21 belonging to family | |
| | Podoviridae genus N4-like virus | |
| | isolated in Japan” Microbiol | |
| | Immunol (2016) vol. 60 pp 64. | |
| JBD4 and | Phee A. et al., “Efficacy of | Pseudomonas |
| JBD44a | bacteriophage treatment on | aeruginosa |
| | Pseudomonas aeruginosa | |
| | biofilms.” J Endod. 2013 | |
| | March; 39(3) | |
| φMR299-2 | Alemayehu D. et al., | Pseudomonas |
| and φNH-4 | “Bacteriophages φMR299-2 and | aeruginosa |
| | φNH-4 can eliminate | |
| | Pseudomonas aeruginosa in the | |
| | murine lung and on cystic fibrosis | |
| | lung airway cells.” MBio. 2012 | |
| | Mar. 6; 3(2) | |
| DMS3, JBD3, | Pawluk A. etal., “A | Pseudomonas |
| JBD5, JBD8, | new group of | aeruginosa |
| JBD16C, JBD23, | phage anti-CRISPR | |
| JBD24, JBD26, | genes inhibits | |
| JBD30, JBD32, | the type I-E | |
| JBD33, | CRISPR-Cas system | |
| JBD35CC, | of Pseudomonas | |
| JBD47, JBD59a, | aeruginosa.” | |
| JBD60a, JBD66, | MBio. 2014 Apr. | |
| JBD69, JBD79, | 15; 5(2) | |
| JBD88a, | | |
| JBD93a, | | |
| JBD95b, | | |
| D3112, MP22, | | |
| MP29 | | |
| JG068 | Lynch K.H., et al., “Genomic | Burkholderia |
| | charcerization of JG068, a novel | cenocepacia |
| | virulent podovirus active against | |
| | Burkholderia cenocepacia” | |
| | (2013) vol. 14, pp 574. | |
| PAS-1 | Kim et al., “Biological control of | Aeromonas |
| | Aeromonas salmonicida sub sp. | salmonicida |
| | Salmonicida infection in rainbow | |
| | trout (Oncorhynchus mykiss) | |
| | using Aeromonas phage PAS-1” | |
| | (2015) vol 62, pp 81. | |
| PY100 | Schwudke et al., “Broad-host- | Yersinia |
| | range Yersinia phage Py100: | enterocholitica, |
| | genome sequence, proteome | Yersinia |
| | analysis of virions, and DNA | pseudotuberculosis, |
| | packaging strategy” J. Bacteriol. | and Yersinia pestis |
| | (2008) vol. 190 pp 332. | |
| φMR11 | Rashel et al., “Efficient | Staphylococcus |
| | elimination of multidrug resistant | aureus |
| | Staphylococcus aureus by cloned | |
| | lysin derived from bacteriophage | |
| | Phi MR11: J. Infec Dis. (2007) | |
| | vol. 196 pp 12437. | |
| SalmoFresh ™ | Sharma et al., “Efficacy of Lytic | Salmonella |
| | Bacteriphage Preparation in | Enteritidis, |
| | Reducing Salmonella In Vitro, on | Salmonella |
| | Turkey Breast Cutlets, and on | Heidelberg, |
| | Ground Turkey” J. Food Prot. | Salmonella |
| | (2015) vol 78 pp 1357. | Kentucky, and |
| | | Salmonella |
| | | Typhimurium |
| phiCDHM1, | Janet Y. Nale, et al. | Clostridium |
| phiCDHM2, | “Bacteriophage | difficile |
| phiCDHM5, | Combinations | |
| phiCDHM6 | Significantly | |
| | Reduce Clostridium | |
| | difficile Growth In Vitro and | |
| | Proliferation In Vivo.” | |
| | Antimicrob | |
| | Agents Chemother. 2016 | |
| | February; 60(2):968-981 | |
| EFDG1 | Khalifa et al., | Enterococcus |
| | Targeting Enterococcus faecalis | faecalis |
| | biofilms with phage | |
| | therapy. Appl | |
| | Environ Microbiol. 2015 April; | |
| | 81(8): 2696-2705. | |

### H. Formulations

In some embodiments, compounds for inhibiting/activating QS may be manufactured ex vivo (e.g., via a small or large scale biomanufacturing process) and purified for administration as a pharmaceutical composition to be administered to recipients. It is presumed that the compound exhibits sufficient stability for administration. In other embodiments, compounds may be administered in combination with phages, e.g., phage therapy may be administered prior to administration of QS inhibiting/activating compounds, at or about the same time as the QS inhibiting/activating compounds, or subsequent to administration of QS inhibiting/activating compounds. These compositions may be administered to, including but not limited to, a patient with antibiotic-resistant infections, to surfaces for disinfection or decontamination, or prophylactically to prevent infection for devices that are implanted into patients.

A pharmaceutical composition (e.g., formulation) may comprise, in addition to the QS compound(s) and/or phages used in phage therapy, one or more pharmaceutically acceptable carriers, adjuvants, excipients, diluents, fillers, buffers, stabilizers, preservatives, lubricants, or other materials well known to those skilled in the art. Suitable materials will be sterile and pyrogen-free, with a suitable isotonicity and stability. Examples include sterile saline (e.g. 0.9% NaCl), water, dextrose, glycerol, ethanol or the like or combinations thereof. Such materials should be non-toxic and should not interfere with the efficacy of the active compound. The precise nature of the carrier or other material will depend on the route of administration, which may be by bolus, infusion, injection or any other suitable route, as discussed herein. Suitable materials will be sterile and pyrogen free, with a suitable isotonicity and stability. Examples include sterile saline (e.g. 0.9% NaCl), water, dextrose, glycerol, ethanol or the like or combinations thereof. The composition may further contain auxiliary substances such as wetting agents, emulsifying agents, pH buffering agents or the like.

Suitable carriers, excipients, etc. can be found in standard pharmaceutical texts, for example, Remington's Pharmaceutical Sciences, 18th edition, Mack Publishing Company, Easton, Pa., 1990.

The term “pharmaceutically acceptable” as used herein pertains to compounds, materials, compositions, and/or dosage forms which are, within the scope of sound medical judgment, suitable for use in contact with the tissues of a subject (e.g. human, animal or plant) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

In some embodiments, the compositions described herein may be provided in a lyophilized form for reconstitution prior to administration. For example, lyophilized reagents may be re-constituted in sterile water and mixed with saline prior to administration to a subject.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols, any of which is suitable for administration of the compositions described herein.

Optionally, other therapeutic or prophylactic agents may be included in a pharmaceutical composition or formulation.

Treatment may include any treatment and therapy, whether of a human or an animal (e.g. in veterinary applications) or plant, in which some desired therapeutic effect is achieved, for example, the inhibition or delay of the progress of the condition (e.g., a bacterial infection), and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment as a prophylactic measure (i.e. prophylaxis) is also included. For example, a subject susceptible to or at risk of the occurrence or re-occurrence of infection may be treated as described herein. Such treatment may prevent or delay the occurrence or re-occurrence of an infection e.g., a bacterial infection in the recipient/patient. Present invention embodiments may also be used for prophylactic treatment of domesticated animals (e.g., cattle, chickens, sheep, pigs, etc.), as an alternative to antibiotics, to prevent the spread of bacterial infections. Present invention embodiments may also be used for prophylactic treatment of plants and in aquaculture to prevent the spread of bacterial infections.

The term “therapeutically-effective amount” as used herein, pertains to that amount of an active compound, or a combination, material, composition or dosage form comprising an active compound, which is effective for producing some desired therapeutic effect, commensurate with a reasonable benefit/risk ratio.

In some embodiments, compounds or compositions in combination with phage therapy are administered to the patient in a therapeutically effective amount, an amount effective to overcome or destroy the infection (e.g., bacterial infection, microbial infection, etc).

It will be appreciated that appropriate dosages of the active compounds or agents can vary from patient to patient. Determining the optimal dosage will generally involve the balancing of the level of therapeutic benefit against any risk or deleterious side effects of the administration. The selected dosage level will depend on a variety of factors including, but not limited to, the route of administration, the time of administration, the rate of excretion of the active compound, other drugs, compounds, and/or materials used in combination, and the age, sex, weight, condition, general health, and prior medical history of the patient. The amount of active compounds and route of administration will ultimately be at the discretion of the physician, although generally the dosage will be to achieve concentrations of the active compound at a site of therapy without causing substantial, harmful, or deleterious side-effects.

In general, a suitable dose of the QS inhibitor/agonist or the QS inhibitor/agonist in combination with phage therapy is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

Preferred modes of administration of phage therapy include oral administration, in tablet or liquid formulation; local or topical administration, to skin, ears, eyes, nasal mucosa, etc., as aerosols or intrapleural injections; as well as intravenously.

Administration in vivo can be effected in one dose, continuously or intermittently (e.g., in divided doses at appropriate intervals). Methods of determining the most effective means and dosage of administration are well known to those of skill in the art and will vary with the formulation, the purpose, the target, and the subject being treated with the composition. Single or multiple administrations can be carried out with the dose level and pattern of administration being selected by the physician.

Administration of a QS inhibitor/agonist and phage therapy may be simultaneous, separate or sequential. By “simultaneous” administration, it is meant that the QS inhibitor/agonist and the phage therapy are administered to the subject in a single dose by the same route of administration at or about the same time.

By “separate” administration, it is meant that the QS inhibitor/agonist and the phage therapy are administered to the subject by two different routes of administration which occur at the same time. This may occur for example wherein one component is administered by infusion or parenterally and the other is given orally during the course of the infusion or parenteral administration.

By “sequential” it is meant that the QS inhibitors/agonists and phage therapy are administered at different points in time, provided that the activity of the first administered agent is present and ongoing in the subject at the time the second agent is administered, and so forth. For example, the QS inhibitory (or activating) compound may be administered first, such that QS is suppressed (or activated), followed by administration of phage therapy to destroy the weakened bacterial cells. Preferably, a sequential dose will occur such that the second of the two agents is administered within 48 hours, preferably within 24 hours, such as within 12, 6, 4, 2 or 1 hour(s) of the first agent.

Multiple doses of the QS inhibitor/agonist and/or the phage therapy may be administered, for example 2, 3, 4, 5 or more than 5 doses may be administered according to the techniques presented herein. The administration of the QS inhibitor/agonist and/or the phage therapy may continue for sustained periods of time. For example, treatment with QS inhibitor/agonist and/or the phage therapy may be continued for at least 1 week, at least 2 weeks, at least 3 weeks, at least 1 month or at least 2 months. Treatment with the QS inhibitor/agonist and/or the phage therapy may be continued for as long as is necessary to achieve complete eradication of the bacterial infection.

In preferred embodiments, administration of the QS inhibitors/agonists will precede administration of phage therapy, in order to sensitize the bacterial cells to treatment with phages.

The active compounds or compositions comprising the active compounds may be administered to a subject by any convenient route of administration, whether systemically/peripherally or at the site of desired action, including but not limited to, oral (e.g. by ingestion); and parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. Other routes such as intraperitoneal, subcutaneous, transdermal, oral, nasal, intramuscular or other convenient routes are also included.

The pharmaceutical compositions comprising the active compounds may be formulated in suitable dosage unit formulations appropriate for the intended route of administration.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for parenteral administration (e.g. by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilizers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Compositions comprising a QS inhibitor/agonist and/or phage therapy may be prepared in the form of a concentrate for subsequent dilution, or may be in the form of divided doses ready for administration. Alternatively, the reagents may be provided separately within a kit, for mixing prior to administration to a human or animal subject.

The QS inhibitor/agonist and/or the phage therapy may be administered alone or in combination with other treatments, either simultaneously or sequentially dependent upon the individual circumstances. For example, the QS inhibitor/agonist and/or the phage therapy as described herein may be administered in combination with one or more additional antimicrobial compounds.

Various embodiments are disclosed above for a QS inhibitor/agonist and/or phage therapy for administration to a patient. Aspects and embodiments of the invention relating to the QS inhibitor/agonist and/or the phage therapy and optionally one or more other agents disclosed above include administration of the compounds or agents separately (sequentially or simultaneously) or in combination (co-formulated or mixed). For each aspect or embodiment, the specification further discloses a composition comprising the QS inhibitor/agonist and/or the phage therapy and optionally one or more other antimicrobial compounds co-formulated or in admixture with each other and further discloses a kit or unit dose containing the QS inhibitor/agonist and/or the phage therapy. Optionally, such compositions, kits or doses further comprise one or more carriers in admixture with or co-packaged for formulation prior to administration to an individual.

Suitable QS inhibitors/agonists and/or phage therapies for the treatment of bacterial infections are described mutatis mutandis above.

### I. Combination Therapy with Antibiotics or Other Anti-Microbials

The compositions described herein may be administered in combination with one or more antimicrobials, such as antibiotics. In some embodiments, the antibiotic may be selected from the following families of antibiotics, including but not limited to: penicillins, cephalosporins, macrolides, quinolones, tetracyclines, and 3-lactams etc. (Nursing 2001 Drug Handbook, (2001), p. 24-214.)

Examples of antibiotic compounds from the β-lactam family include: Amidinocillin, Amoxicillin, Ampicillin, Apalcillin, Aspoxicillin, Azidocillin, Azlocillin, Aztreonam, Bacampicillin, Benzylpenicillic acid, Carbenicillin, Carfecillin, Carindacillin, Carumonam, Cefaclor, Cefadroxil, Cefamandole, Cefatrizine, Cefazedone, Cefazolin, Cefbuperazone, Cefixime, Cefmenoxime, Cefotaxime, Ceftizoxime, Cefmetazole, Cefminox, Cefodizime, Cefonicid, Cefoperzone, Ceforanide, Cefotetan, Cefotiam, Cefoxitine, Cefpimizole, Cefpiramide, Cefpodoxime proxetil, Cefroxadine, Cefsulodin, Ceftazidime, Cefteram, Ceftezole, Ceftibuten, Ceftiofur, Ceftizoxime, Ceftriaxone, Cefuroxime, Cefuzonam, Cephacetrile, Cephalexin, Cephaloglycin, Cephaloridine, Cephalosporin C, Cephalothin, Cephapirin, Cepharanthine, Cephradine, Clometocillin, Cloxacillin, Cyclacillin, Dicloxacillin, Diphenenicillin, Epicillin, Fenbenicillin, Flomoxef, Floxacillin, Hetacillin, Imipenem, Lenampicillin, Metampicillin, Methicillin, Mezlocillin, Moxolactam, Nafcillin, Oxacillin, Penamecillin, Penamethate hydriodide, Penicillin, Penimepicycline, Phenethicillin, Piperacillin, Pivampicillin, Pivcefalexin, Propicillin, Quinacillin, Sulbenicillin, Sulfazecin, Talampicillin, Temocillin, Ticarcillin, and Tigemonam.

Examples of antibiotic compounds from the penicillin family include: amoxcillin/clavulanate potassium, amoxicillin trihydrate, ampicillin, ampicillin sodium, ampicillin trihydrate, ampicillin sodium/sulbactam sodium, cloxacillin sodium, dicloxacillin sodium, mezlocillin sodium, nafcillin sodium, oxacillin sodium, penicillin G benzathine, penicillin G potassium, penicillin G procaine, penicillin G sodium, penicillin V potassium, piperacillin sodium, piperacillin sodium/tazobactam sodium, ticarcillin disodium, and ticarcillin disodium/clavulanate potassium.

Examples of antibiotic compounds from the quinolone family include: Cinoxacin, Ciprofloxacin, Enoxacin, Fleroxacin, Flosequinan, Flumequine, Pomefloxacin, Nalidixic acid, Norfloxacin, Ofloxacin, Oxolinic acid, Pefloxacin, Pipemidic acid, Piromidic acid, Rosoxacin, and Sparfloxacin. Examples of antibiotic compounds from the fluoroquinolone family include: alatrofloxacin mesylate, ciprofloxacin, enoxacin, levofloxacin, lomefloxacin hydrochloride, nalidixic acid, norfloxacin, ofloxacin, sparfloxacin, and trovafloxacin mesylate.

Examples of antibiotic compounds from the cephalosporin family include: cefaclor, cefadroxil, cefazolin sodium, cefdinir, cefepime hydrochloride, cefixime, cefmetazole sodium, cefonicid sodium, cefoperazone sodium, cefotaxime sodium, cefotetan disodium, cefoxitin sodium, cefpodoxime proxetil, cefprozil, ceftazidime, ceftibuten, ceftizoxime sodium, ceftriaxone sodium, cefuroxime axetil, cefuroxime sodium, cephalexin hydrochloride, cephalexin monohydrate, cephradine, and loracarbef.

Examples of antibiotic compounds from the tetracycline family include: demeclocycline hydrochloride, doxycycline calcium, doxycycline hyclate, doxycycline hydrochloride, doxycycline monohydrate, minocycline hydrochloride, and tetracycline hydrochloride. The at least one sulfonamide can be selected from co-trimoxazole, sulfadiazine, sulfamethoxazole, sulfisoxazole, and sulfisoxazole acetyl.

Examples of antibiotic compounds from the macrolide family include: azithromycin, clarithromycin, dirithromycin, erythromycin base, erythromycin estolate, erythromycin ethylsuccinate, erythromycin lactobionate, and erythromycin stearate.

Other types of antibiotics include: aztreonam, bacitracin, chloramphenicol sodium sucinate, clindamycin hydrochloride, clindamycin palmitate hydrochloride, clindamycin phosphate, imipenem and cilastatin sodium, meropenem, nitrofurantoin macrocrystals, nitrofurantoin microcrystals, quinupristin/dalfopristin, spectinomycin hydrochloride, trimethoprim, and vancomycin hydrochloride.

Synergy may be observed when administering a combination of a QS inhibitor/agonist and a phage therapy, and an antibiotic or other antimicrobial. In some embodiments, a 10%, a 20%, a 30%, a 40%, a 50%, a 60%, a 70%, an 80% a 90% and so forth, synergistic effect may be observed with the combination as compared to treatment with a QS inhibitor/agonist, phage therapy, or antibiotic alone.

### J. Cell Culture

The cell types of the present invention embodiments may be cultured in any manner known in the art. Methods of bacterial cell culture are described in, e.g., Ball, A. S., Bacterial Cell Culture: essential data, Wiley (1997), the contents of which are incorporated herein in their entirety by reference. General cell culture techniques, lines, and systems suitable for use with present invention embodiments are also described in, e.g., Doyle, A., Griffiths, J. B., Newell, D. G., (eds.) Cell and Tissue Culture: Laboratory Procedures, Wiley (1998), the contents of which are incorporated in their entirety herein by reference. Methods of handling phages are described in, e.g., Holm, T., Application of Bacteriophages in Clinical Medicine, OmniScriptum GmbH & Co. KG (2015), the contents of which are incorporated in their entirety by reference herein.

Appropriate growth conditions for bacterial cells in cell culture are well known in the art. Cell culture media generally includes essential nutrients and, optionally, additional elements such as growth factors, salts, minerals, vitamins, platelet-rich plasma, etc., selected according to the cell type(s) being cultured. In some embodiments, particular ingredients are selected to enhance cell growth, differentiation, secretion of specific proteins, etc.

QS serves as a mechanism to protect bacteria from phage infection. By inhibiting (or activating) QS, bacterial virulence and other bacterial defenses, bacteria become vulnerable to natural killing by the recipient immune system as well as by phages. By administering QS inhibitors that inhibit not only previously known QS functions but also CRISPR-Cas immune activity, QS inhibitors additionally sensitize the bacteria to make the bacteria more susceptible to phage therapy. Without a functional CRISPR-Cas system, the bacteria cannot achieve adaptive immunity by inserting a fragment of the phage into the CRISPR-Cas genomic loci and this minimizes the risk of the bacteria gaining adaptive immunity towards administered phage therapy. In other systems such as cholera, administration of QS agonists represses pathogenicity and biofilm formation. Thus, inhibition or activation of bacterial QS represents an innovative approach for developing novel antimicrobial agents.

Present invention embodiments include novel antimicrobial therapies. QS inhibitors (or QS agonists) and phage therapy, separately or in combination, may be administered to eradicate pathogenic bacteria, exhibiting synergistic effects in their anti-microbial actions, while minimizing emerging therapy resistance. QS inhibitors (or QS agonists) or phage therapy may also be used in combination with other agents. Accordingly, pathogenic bacteria can be eliminated without selecting for treatment-resistant strains of bacteria. The techniques presented herein are broadly applicable to medical therapy, industrial, and agricultural use.

### EXAMPLES

### Example 1. Cas Expression in OS Deficient Strains

Using known molecular biology techniques, P. aeruginosa PA14 (the wild type strain) was genetically modified to create mutant strains lacking one or more genes involved in QS. For example, mutant strains ΔlasR, ΔrhlR, ΔlasR ΔrhlR, ΔlasI ΔrhII, and ΔlasI ΔrhII ΔpqsA, which lacked a functional gene product encoded by lasR, rhlR, lasR rhlR, lasI rhII and lasI rhII pqsA were generated.

In one example protocol, P. aeruginosa PA14 and mutants were grown overnight at 37° C. with shaking in LB broth. Cultures were back diluted 1:100 and grown to the indicated OD₆₀₀ in the presence or absence of DMSO, 3-O—C₁₂-HSL, C₄—HSL (Sigma) or Baicalein (Cayman Chemical) at the specified concentrations. LB was supplemented with 50 μg/ml Gentamicin where appropriate.

For Quantitative Real Time-PCR (qRT-PCR), bacteria were harvested at the indicated OD₆₀₀. RNA was purified using Trizol (Ambion), DNAse treated using DNA-free (Ambion), cDNA was synthesized using SuperScript® III Reverse Transcriptase (Invitrogen) and quantified using PerfeCTa® SYBR® Green FastMix®, Low ROX (Quanta Biociences).

Quantitative Real Time-PCR (qRT-PCR) was utilized to determine the relative expression of cas3 in each of these mutant strains. Additionally, 100 μM 3-O—C₁₂-HSL and 100 μM C₄-HSL, which are AIs that rescue cas3 expression, were added to the cell culture comprising the mutant strain ΔlasI ΔrhII. It is understood that other concentrations of 3-O—C₁₂-HSL and C₄—HSL are also suitable, e.g., the concentrations may be in the range of 2 μM to 100 μM, from 10 μM to 100 μM or from 2 μM to 10 μM. In some embodiments, 2 μM of 3-O—C₁₂-HSL and 10 μM of C₄-HSL were used. The expression profiles for cas3 for each strain were then normalized based on expression of 5S ribosomal RNA. The expression data for wild type (WT) Pseudomonas aeruginosa PA14, QS mutants (ΔlasR, ΔrhlR, ΔlasR ΔrhlR and ΔlasI ΔrhII) grown in the presence of DMSO solvent (as a control), and QS mutant (ΔlasI ΔrhII) grown in the presence of AIs were plotted in the form of a histogram in FIG. 1. The mutant bacterial strains deficient in QS were shown to express lower levels of cas3 as compared to the WT strain. Rescue with AIs was shown to restore cas3 expression to WT levels. Accordingly, molecules involved in QS (such as lasR, rhlR, lasI, and rhII and AIs) were shown to regulate the expression of the cas3 genes (nuclease and helicase).

### Example 2A. Transformation Assay to Assess Efficiency of Transformation

Bacteria were grown to the appropriate OD₆₀₀, washed twice at RT in 300 mM sucrose, and electroporated with 1 μg empty vector plasmid pHERD30T or pCR2SP1 containing a phage-derived sequence specifically targeted for degradation by CRISPR-Cas (Bondy-Denomy, Pawluk et al. 2013). 1 ml LB was added and the bacteria were grown 1 h at 37° C. with shaking, after which they were plated on selective medium, e.g., LB medium containing 50 μg/ml gentamicin, and incubated overnight at 37° C. Bacterial colonies were counted using Image Quant Las 4000 and Image Quant TL software (GE Healthcare). Colony forming units (CFU)/ml were quantified and the efficiency of transformation was calculated as the percentage of colonies transformed by pCR2SP1 compared to those transformed by the empty vector control pHERD30T.

This assay utilizes a plasmid as a proxy for a phage, to quantify the frequency with which a foreign genetic material is identified and targeted for degradation by the host bacterial immune system. For bacterial cells having a functional CRISPR-Cas system, the plasmid will be targeted for destruction, and no colonies will grow on the plate, as antibiotic resistance has been lost.

For cells lacking a CRISPR-Cas system or having an impaired or partially functioning CRISPR-Cas system, the electroporated plasmid evades destruction by the bacterial host and is able to replicate. In this case, the plasmid is able to confer antibiotic resistance to the host bacterial cell and colonies are formed on selective medium.

In FIG. 2A, results of a transformation assay are shown. The efficiency of transformation (EOT), which involves the introduction of foreign DNA (e.g., a plasmid proxy targeted for elimination by the CRISPR-Cas system), was determined for WT bacterial strain P. aeruginosa PA14, CRISPR-Cas deficient strain (ΔCRISPR Δcas), QS deficient strain (ΔlasI ΔrhII), and QS deficient strain (ΔlasI ΔrhII) rescued by AIs 3-O—C₁₂-HSL and C₄—HSL.

In the WT strain P. aeruginosa PA14, the efficiency of transformation was determined to be 0.7%, indicating that the bacterial immune system was 99.3% efficient at eliminating foreign DNA via the CRISPR-Cas system. In the absence of CRISPR-Cas (ΔCRISPR Δcas strain), the bacterium failed to eliminate the foreign DNA. QS mutant (ΔlasI ΔrhII) had more than a 10-fold increase in EOT as compared to WT, indicating that this QS mutant was more than 10-fold less efficient in eliminating foreign DNA. Adding 3-O—C₁₂-HSL and C₄—HSL to the QS mutant (ΔlasI ΔrhII) restored immune activity to nearly WT levels. Accordingly, these results demonstrated that cells deficient in QS have a corresponding deficiency in elimination of foreign genetic material (and therefore, have an increased susceptibility to destruction by phages) by the CRISPR-Cas system.

QS control of CRISPR-Cas provided P. aeruginosa up to a 10-fold increase in resistance to incoming foreign DNA, as shown by this technique.

### Example 2B: Plaque Assay to Assess Efficiency of Plaquing

PA14 was adapted to phage JBD44a and a single spacer in CRISPR 2 was confirmed to match JBD44a. This strain was used to generate an adapted QS mutant (ΔlasI ΔrhII ΔpqsA). Bacteria were grown to the appropriate OD₆₀₀. 100 μl culture was mixed with 55° C. soft agar and overlayed on an LB plate. A virulent variant of JBD44a, JBD44a^{vir} (at a concentration of 10¹⁰ plaque forming units pr ml) was spotted in 5-fold dilutions on the solidified soft agar and incubated at 37° C. over night and imaged.

This assay utilizes a CRISPR-targeted phage, to quantify the efficiency with which a phage is able to kill bacteria. For bacterial cells having a functional CRISPR-Cas system, the phage will be targeted for destruction, and the cells will survive and no plaque will form on the plate where the phage was spotted. For high titers of phage and/or cells with low CRISPR-Cas activity, the phage will kill the cells and form a plaque.

In FIG. 2B, results of a plaque assay is shown. The efficiency of plaquing (EOP), which involves phage-mediated killing (e.g. a phage targeted for elimination by the CRISPR-Cas system), was determined for an adapted bacterial strain P. aeruginosa PA14 and an adapted QS mutant (ΔlasI ΔrhII ΔpqsA).

In the adapted strain P. aeruginosa PA14, EOP was approximately 10-fold lower than that of the adapted QS mutant (ΔlasI ΔrhII ΔpqsA), indicating that this QS mutant was 10-fold less susceptible to phage killing.

QS control of CRISPR-Cas provided P. aeruginosa approximately 10-fold increase in resistance to phage killing, as shown by this technique.

### Example 3: Methods for Measuring QS, Biofilm Production, Biofilm Streamer Production and/or Virulence Factor Production

Methods for measuring QS, biofilm production, biofilm streamer production and/or virulence factor production have been reported in the literature and are herein incorporated by reference in their entirety. For example, see, Kim M K et al. “Filaments in curved streamlines: Rapid formation of Staphylococcus aureus biofilm streamers,” New J Phys. 2014 Jun. 26; 16(6):065024; Ng W L, et al., “Broad spectrum pro-quorum-sensing molecules as inhibitors of virulence in vibrios,” PLoS Pathog. 2012; 8(6); and O'Loughlin C T, “A Quorum-Sensing Inhibitor Blocks Pseudomonas aeruginosa Virulence And Biofilm Formation,” PNAS (2013) Oct. 29; 110(44): 17981-6. Compositions of the invention can be tested in any of these published protocols.

### Example 4: Methods for Determining Adaptation to the CRISPR Genomic Locus

A P. aeruginosa PA14 ΔlasI ΔrhII QS deficient strain was transformed with foreign DNA (e.g., a CRISPR-targeted plasmid such as pCR2SP1, as described above). A single colony from the transformation was re-streaked on selective medium, e.g., LB medium containing 50 μg/ml gentamicin and either DMSO (as a control), AIs (2 μM 3-O—C₁₂-HSL+10 μM C₄-HSL), or both AIs and a QS inhibitor (2 μM 3-O—C₁₂-HSL+10 M C₄-HSL+100 μM Baicalein QS inhibitor) and incubated at 37° C. O/N. Single colonies were tested for integration of new immunity spacers against the CRISPR-targeted plasmid by PCR using iProof High-Fidelity DNA Polymerase (Bio-Rad) and primers CR2SP check F+R upstream of the CRISPR2 region and in the second spacer. Thus, PCR primers were designed to amplify the genomic region in which the new spacer was present. Primers targeting a sequence upstream of the CRISPR-Cas region and targeting the second spacer region were designed, and PCR amplification was utilized to test for the presence of the new inserted spacer (a portion of the plasmid sequence inserted between the CRISPR locus and spacers). The PCR reaction was then subjected to gel electrophoresis; DNA lane markers were added for reference, as shown in the first lane of the gel of FIG. 3.

Addition of AIs, as compared to the DMSO control, increases the fraction of cells in the colony that have adapted, while addition of AIs in combination with a QS inhibitor (e.g., Baicalein) blocks the positive effect of AIs on CRISPR adaptation. Thus, QS increases the chance that a given bacterium will acquire a new CRISPR spacer and thereby increase its ability to successfully eliminate the foreign plasmid/phage. Conversely, QS inhibition minimizes the risk of the bacterium gaining adaptive immunity towards the foreign plasmid/phage, which effectively makes the bacterium less likely to become resistant to plasmid/phage.

### Example 5. Small Molecule Inhibitors of Receptors Involved in OS

A two plasmid E. coli reporter system was constructed, in which LasR or RhlR was expressed under an arabinose inducible promoter (e.g., LasR or RhlR was cloned into a pBAD-A vector and expressed upon addition of arabinose) in the presence of a LasR-dependent or RhlR-dependent promoter fused to luciferase (e.g., cloned into a pCS26 vector). Upon the addition of arabinose and in the presence of AI, LasR or RhlR were expressed, formed a complex with the cognate AI, underwent dimerization, and activated transcription of the reporter construct. All potential inhibitors were tested at 100 μM. The percent activity was reported relative to wild-type (100%).

Although this example uses a two plasmid reporter system, present invention embodiments are not intended to be restricted to such examples as, e.g., both LasR/RhlR and their cognate ligands may be present on the same plasmid. Reagents used in this assay include:

TABLE 5A1

| Reagent | Description | Source |
|---|---|---|
| E. coli | Top10 | Invitrogen |
| strain | Genotype: F-mcrA | |
| | Δ(mrr- | |
| | hsdRMS-mcrBC) | |
| | Φ80lacZΔM15 | |
| | Δ lacX74 recA1 araD139 | |
| | Δ(araleu)7697 galU | |
| | galK rpsL | |
| | (StrR) endA1 nupG | |
| LasR | 6xHIS-lasR in pBAD-A | Thermo |
| reporter | (ampicillin resistant) vector; | Fisher |
| construct | plasB-luxCDABE in pCS26 | |
| with | (kanamycin resistant) in TOP10. | |
| arabinose | Control strain: pTac-luxCDABE | |
| induction | in pCS26 in TOP10 (lacks locI | |
| | repression − constitutively | |
| | active) (kanamycin resistant) | |
| Growth | LB with 100 ug/ml amp and kan | Fisher |
| Media | 50 ug/mL for solid and liquid | Antibiotic (Sigma) |
| | media | stocks were |
| | | made and stored at |
| | | −20° C. Amp |
| | | was made as a 100 |
| | | mg/ml stock. |
| | | Kan was made as |
| | | a 50 mg/ml |
| | | stock. |
| Agonist | 3-O—C₁₂-HSL (AI) is stored at | Semmelheck Lab |
| standard | 100 mM in DMSO (Sigma) at— | (available |
| | 80° C. until used, then stored at | through Sigma) |
| | −20° C. at 1 μM. | |
| Inhibitor | mBTL is stored as DMSO stock | Semmelhack |
| standard | at 100 mM at −20° C.. Master | lab |
| | stock stored at −80° C.. | Princeton |
| | Inhibitors at screening library | Chemistry |
| | were stored at 10 mM at −80 C. | Department |
| | Solids purchased from the | Various (TCI, |
| | screening hits were made to 10 | ChemBridge, |
| | mM and stored at −20° C.. | ChemDiv, Cayman |
| | | Chemicals, |
| | | Santa Cruz |
| | | Chemicals, ALFA |
| | | AESA) |
| Arabinose | 10% w/v in H₂O (autoclaved, DI | Sigma |
| inducer | water made) stored at room | |
| stock | temperature | |

TABLE 5A2

| Reagent | Description | Source |
|---|---|---|
| E. coli | Top10 | Invitrogen |
| strain | Genotype: F-mcrA Δ( mrr- | |
| | hsdRMS-mcrBC) | |
| | Φ80lacZΔM15 | |
| | Δ lacX74 recA1 araD139 | |
| | Δ(araleu)7697 galU galK rpsL | |
| | (StrR) endA1 nupG | |
| RhlR | 6xHIS-rhlR in pBAD-A | Thermo |
| reporter | (ampicillin resistant) vector; | Fisher |
| construct | prhlA-luxCDABE in pCS26 | |
| with | (kanamycin resistant) in TOP10. | |
| arabinose | Control strain: pTac-luxCDABE | |
| induction | in pCS26 in TOP10 (lacks locI | |
| | repression − constitutively | |
| | active) (kanamycin resistant) | |
| Growth | LB with 100 ug/ml amp and kan | Fisher |
| Media | 50 ug/mL for solid and liquid | Antibiotic (Sigma) |
| | media | stocks were |
| | | stored at −20° C.. |
| | | Amp was |
| | | prepared as a 100 |
| | | mg/ml stock. |
| | | Kan was prepared |
| | | as a 50 mg/ml |
| | | stock. |
| Agonist | C4-HSL (AI) was stored at 100 | Semmelheck Lab |
| standard | mM in DMSO (Sigma) at | (available |
| | −80° C. until used, then | through Sigma) |
| | stored at −20° C.. | |
| Inhibitor | mBTL was stored as DMSO | Semmelhack lab |
| standard | stock at 100 mM at −20° C.. | Princeton Chemistry |
| | Master stock at −80° C.. | Department |
| | Inhibitors at screening library | Various (TCI, |
| | were stored at 10 mM at −80° C.. | ChemBridge, |
| | Solid compounds purchased | ChemDiv, Cayman |
| | from the screening hits were | Chemicals, |
| | made to 10 mM and stored at | Santa Cruz Cehmicals, |
| | −20° C.. | ALFA |
| | | AESA) |
| Arabinose | 10% w/v in H₂O (autoclaved, DI | Sigma |
| inducer | water) stored on benchtop | |
| stock | | |

TABLE 5B

| | | | Settings or other |
|---|---|---|---|
| | | | critical notes such |
| | | | as calibration |
| | | | procedures/schedule, |
| | | | plate coatings or |
| | | | pretreatments, |
| Equipment | Description | Source | etc. |
| Bacterial | | | 37° C. for initial growth, |
| incubator | | | used plate |
| | | | shaker set to 30° C. |
| | | | for induction. |
| Pipetting | pipettemen | | |
| equipment/ | | | |
| manual | | | |
| Pipetting | | | |
| equipment/ | | | |
| automated | | | |
| Assay | Envision | Perkin | Temperature of plate |
| detection | 2103 | Elmer | reader was set to |
| equipment | Multilabel | | room temperature. Luminescence |
| | Reader | | aperture was the default 384 plate |
| | | | luminescence aperture with a |
| | | | measurement time of |
| | | | 0.1 seconds and |
| | | | 0 mm distance between plate and |
| | | | detector. OD₆₀₀ reading was done |
| | | | using a photometric 600 filter at |
| | | | 100% light emission with 10 flashes |
| | | | of light at a measurement height of |
| | | | 6.5 mm. |
| Assay | Corning | | TC treated. No additional treatments |
| plates | 3904 TC | | |
| | treated | | |
| Plate filler | Multi | Thermo | Plate size was set to 384 standard |
| | Drop | Scientific | (15 mm), standard tube cassette was |
| | Combi | | selected and set to 20 uL |
| | | | volume, and correct |
| | | | columns for dispensing cells |
| | | | was selected. |

TABLE 5C

| Materials Prep/Scale | |
|---|---|
| for Assay | Prep process |
| Prep of growth | Standard LB prep from powder |
| media for E. coli | |
| Storage of reporter | Picked multiple colonies. Grew them overnight. |
| strain stocks | Stored at −80° C. in 40% |
| | glycerol. Cells were flash |
| | frozen in liquid N₂ and frozen immediately |
| Amplification of | To start cultures, tubes were warmed from the |
| LasR/Rh1R reporter | freezer, and a sample from |
| transfected | the top was obtained by |
| stocks for assay | scraping. The sample was spread |
| scale production | onto an LB plate with Kan and Amp |
| | (same concentrations as used in |
| | liquid culture.) Individual colonies were then |
| | picked and used for starter cultures. Starter |
| | cultures were prepared by growing overnight. |
| | Fresh cultures from a fresh streaked plate were |
| | preferred. Cultures were grown to an OD of |
| | approximately 0.4. If the OD was >1.0, it was |
| | diluted 1:10 and rechecked, as >1.0 the |
| | value was not reliable. |
| Preparation of | Assay plates were dispensed by the Princeton |
| assay plates | University Small Molecule Screening Center. |
| | Liquid stores of small molecule |
| | stocks were kept at −80° C. and 200 nL |
| | were dispensed into 384-well |
| | assay plates 24 hours before use. |
| | Individual 10 point dose response |
| | assays were set up using an HP D300 |
| | Digital Dispenser and D8 |
| | cassettes for drug dispensing. |

A sample experimental protocol for the RhlR reporter (see, e.g., Table 5A2) or for the LasR reporter (see, e.g., Table 5A1).

For the RhlR reporter, strains were re-streaked on LB+appropriate antibiotic before each experiment and grown overnight at 37° C. Three colonies were picked and a 3 mL overnight culture in LB+appropriate antibiotic was started. The culture was grown overnight at 37° C. The overnight culture was back-diluted 1:1000 into fresh LB+appropriate antibiotic in an appropriate volume, depending on the number of plates being screened. Each well in a 384 well plate received about 20 μL of culture. An additional 25 mL of culture was reserved for priming the plate filler. The culture was grown at 37° C. until OD₆₀₀˜0.5, which took about 4 hrs. A 384-well plate was prepared. Stock concentrations of molecules (e.g., small molecule inhibitors) were 10 mM.

For a 10-point dose response for the RhlR reporter, a 1:3 dilution of molecule:DMSO (333 μL of stock molecule, 667 μL of DMSO using a 1 mL pipetteman) was used for accurate dispensing by the robot dispenser. Upon a prompt by the software, the appropriate concentration of each molecule was added. Each well was normalized with a DMSO standard to bring the total % volume of DMSO up to 1% using the robot. Plates were set aside with dispensed drugs until the cells were ready. Once the cells reached an OD₆₀₀ of 0.5, an appropriate amount of cell culture was dispensed for screening into an Erlenmeyer flask. (Extra culture was saved for a negative control.) Arabinose was added to 0.1% final concentration (1:1000 dilution from stock solution), and AI was added to 10 μM (ED75) from a 10 mM stock. The components of the flask were mixed together by swirling. As mentioned previously, other concentrations of AIs may be suitable, e.g., the concentrations may be in the range of 2 uM to 100 uM, from 10 uM to 100 uM or from 2 uM to 10 uM. In some embodiments, 2 nM of 3-O—C₁₂-HSL (for LasR) and 10 uM of C₄-HSL (for RhlR) were used.

A clean plate was filled with water, 70% ethanol, LB, and primed with cultured cells to ensure that initial dispensing led to the appropriate volume of cells being added to the wells. 20 μL of this solution was dispensed into appropriate wells in a 384-well plate that contained small molecule antagonists. 20 μL of this solution was dispensed into wells intended for a positive control that contain only DMSO. To the culture that was saved for negative control, arabinose was added to 0.1%. An equal volume of DMSO was added to the culture, relative to the amount of AI added to the other culture. 20 μL of culture was dispensed into wells intended for a negative control that contained only DMSO and no small molecule QS inhibitors. Plates were incubated at 30° C. in the plate shaker. Plates were read using a 384 LUM/OD 600 setting on the plate reader after 4 hours of induction.

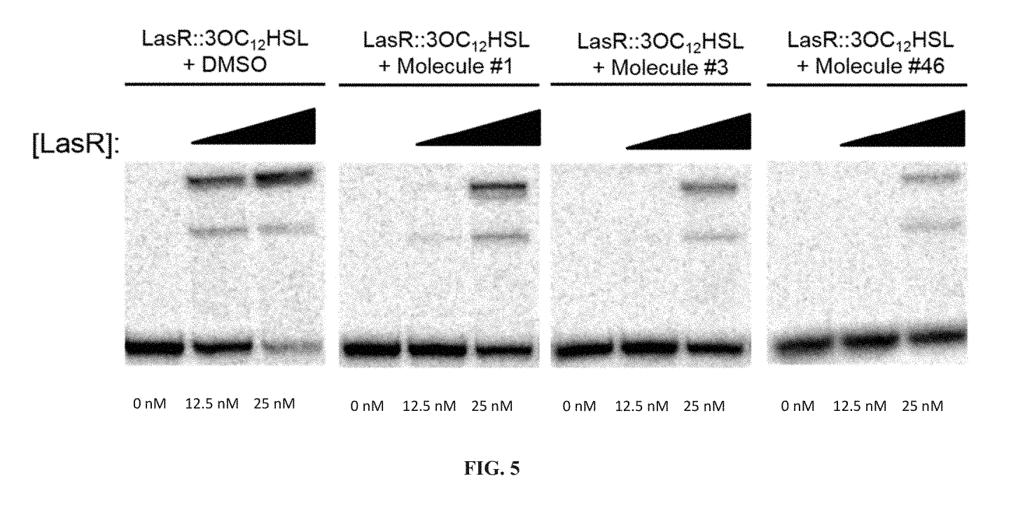

For data analysis, the average and standard deviation for each of the control groups were determined. In a screen, for each data point, the number of standard deviations from the mean was determined to determine if the inhibitor was a “hit”. In a dose response curve, for each data point, the percent inhibition was determined: 1−(X/M), where X=single data point and M=is the mean of the AI only control group. The dose response curve was then plotted as a function of % inhibition (y) v. [inhibitor] on a log scale (x). Dose response curves are shown in FIG. 4, see also, Example 6.

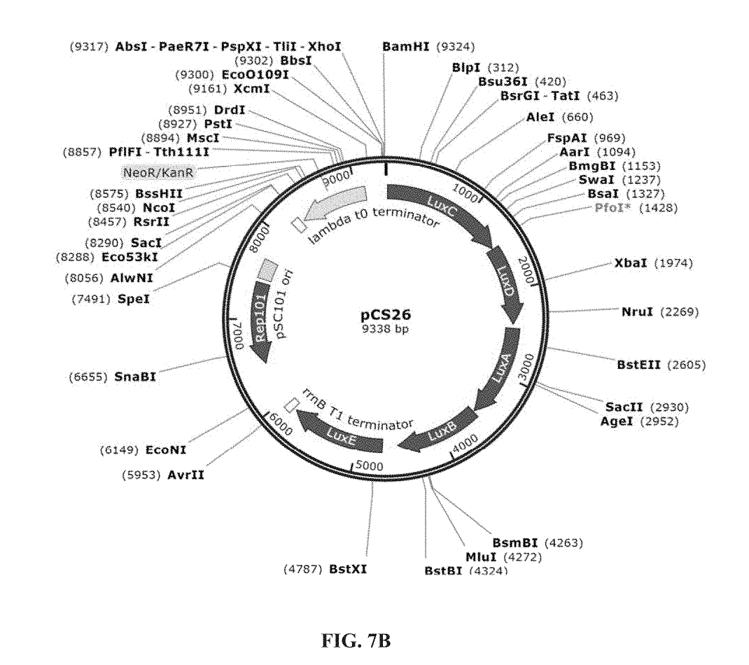

Plasmid maps are shown in FIGS. 7A-7B.

The results are provided in Table 6.

TABLE 6

| | | Percent | Percent |
|---|---|---|---|
| | | Activity | Activity |
| Molecule | | (LasR | (RhlR |
| Number | Chemical Name | reporter) | reporter) |
| 1 | Phloretin | 24.1 | 68.4 |
| 3 | Chrysin | 53.1 | 39.2 |
| 4 | Narigenin | 63.3 | 60.5 |
| 41 | Flavone | Inactive | Inactive |
| 42 | Acacetin | Inactive | Inactive |
| 43 | Apigenin | 82.4 | 84.5 |
| 44 | 5-hydroxyflavone | Inactive | Inactive |
| 45 | 7-hydroxyflavone | Inactive | Inactive |
| 46 | Baicalein | 46.7 | 57.4 |
| 47 | Diosmetin | Inactive | Inactive |
| 48 | Quercetin | 44.4 | 40.2 |
| 49 | 5,7-dimethoxyflavone | Inactive | Inactive |
| 50 | 6-hydroxyflavone | Inactive | Inactive |
| 51 | 5,7,4′-trimethyoxyflavone | Inactive | Inactive |
| 52 | 5,7-dimethoxy-4′hydroxyflavone | Inactive | Inactive |
| 53 | 5,6-dihydroxyflavone | 16.7 | 31.8 |
| 54 | 7,8-dihydroxyflavone | 60.3 | 54.5 |
| 55 | 3,5,7-trihydroxyflavone | Inactive | Inactive |

### Example 6. Small Molecule Inhibitors of Receptors Involved in OS

Using known molecular biology techniques, assays were performed to determine whether binding of the small molecule QS inhibitors and the AIs occurred by a competitive or by another inhibitory mechanism (see, e.g., FIG. 4).

Thus, a 10-point dose response curve with concentrations of AI ranging from 10 pM to 5 nM was generated for a variety of QS small molecule inhibitors. Curve C12 (dotted filled line) corresponds to the absence of inhibitor. The other lines correspond to the presence of a QS inhibitor (100 μM), i.e. #1 corresponds to phloretin, #3 corresponds to chrysin, #46 corresponds to baicalein, and #48 corresponds to quercetin, which were each found to inhibit both LasR and RhlR. Despite increasing the concentration of AIs, LasR-dependent transcription of the reporter remained significantly inhibited at the highest AI dose, indicating that the inhibitors did not compete (competitively bind) with the AI at the ligand-binding pocket. Instead, the inhibitors bind elsewhere on LasR to prevent LasR-dependent transcription.

### Example 7. OS Inhibitors Block Gene Transcription by Preventing Receptor-DNA Binding

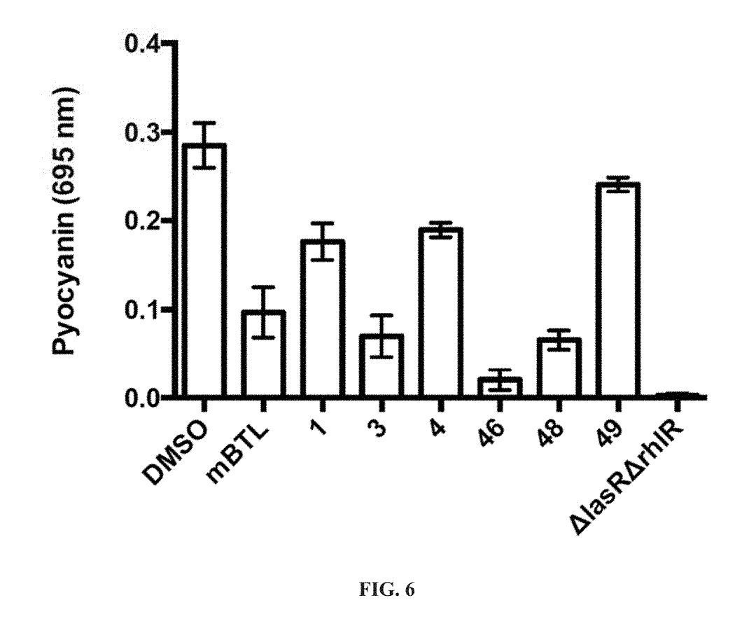

A DNA-binding assay (an electrophoretic mobility shift assay) was performed to assess the DNA-binding ability of LasR bound to an inhibitor (e.g., molecule #1, molecule #3, or molecule #46). Recombinantly expressed and purified LasR bound to the AI (3-O—C₁₂-HSL) was incubated with DMSO (as a control) and 100 M of the inhibitor for 15 min, and then mixed with a radioactively labeled LasR-dependent promoter sequence for 30 min. Three different concentrations of LasR were used for each experiment (0 nM, 12.5 nM, and 25 nM). Results are shown in FIG. 5.

Each of the small molecule QS inhibitors was shown to reduce the affinity of LasR for DNA by at least 50%, indicating that these molecules blocked gene transcription by preventing LasR from binding DNA.

### Example 8. OS Inhibitors Reduce the Level of Virulence Factor Production in P. aeruginosa

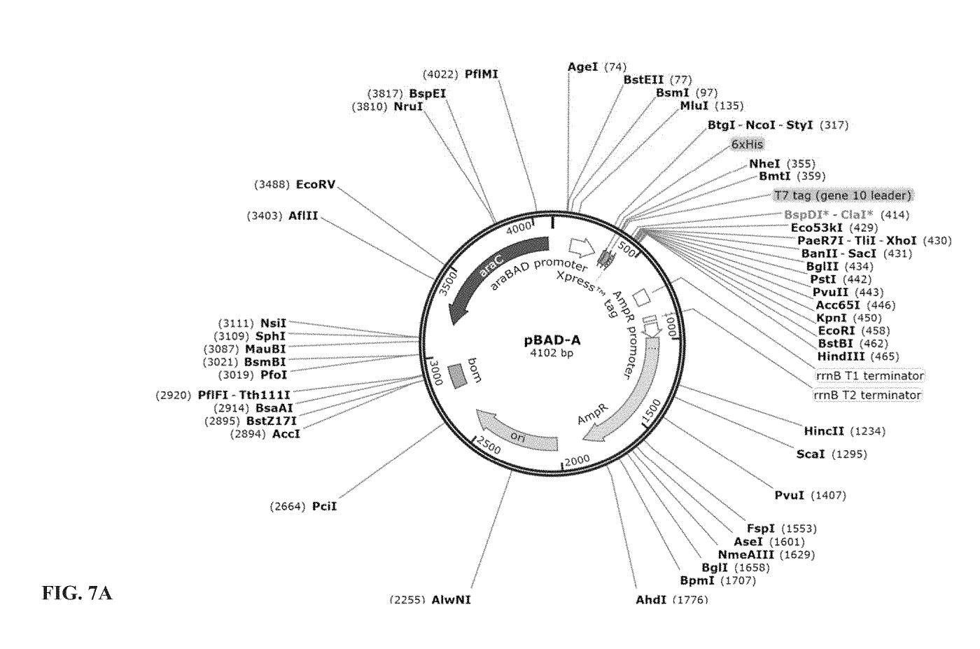

The ability of the small molecule QS inhibitors to reduce virulence factor production by monitoring production of pyocyanin as a representative virulence factor was assessed. Wild-type P. aeruginosa was incubated with the indicated molecules at 100 μM overnight, back diluted, and grown for 18 hrs in the presence of QS inhibitor. These molecules also inhibit pyocyanin to varying degrees, likely dependent on their ability to be metabolized by or bypass the efflux pumps in P. aeruginosa. Results are shown in FIG. 6.

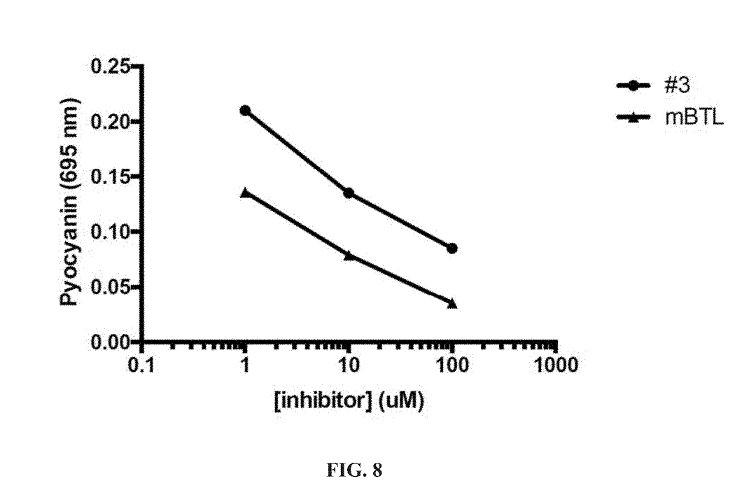

By varying the concentration of QS inhibitors, dose dependence curves were generated. QS inhibitors (e.g., flavonoids) were shown to inhibit pyocyanin in a dose-dependent manner (see, e.g., FIG. 8).

### Example 9. Generation of P. aeruginosa OS Gene Deletion Strains with Bioluminescent Reporters

Deletion strains of key QS genes were created and described according to methods disclosed in Hoyland-Kroghsbo et al. (16). To construct chromosomal deletions in P. aeruginosa PA14, DNA fragments flanking the gene of interest were amplified, stitched together by Gibson assembly, and cloned into pEXG2 (from Joseph Mougous. University of Washington, Seattle) (17, 18). The resulting plasmids were used to transform E. coli SM 10, and subsequently mobilized into PA14 via biparental mating. Exconjugants were selected on LB (Luria-Bertani) containing gentamicin (30 μg/mL) and irgasan (100 μg/mL), followed by recovery of deletion mutants on M9 medium containing 5% (wt/vol) sucrose. Candidate mutants were confirmed by PCR. To construct constitutively bioluminescent strains for mouse infection, the plasmid pUC-miniTn7T-lux-Tp was mobilized into the WT, ΔlasR, ΔlasI, ΔrhlR, and ΔrhlI mutants (19, 20).

### Example 10. High-Throughput Liquid Lysis Assay to Determine Host Infectivity and Cocktail Synergy

An automated, indirect, liquid lysis assay is used to evaluate the activity of a library of purified phages against WT, or ΔlasI ΔrhlI ΔpqsA P. aeruginosa strains, generated as described above according to techniques known in the art (20, 21). Briefly, an overnight culture of each strain is inoculated into the wells of a 96-well plate containing TSB mixed with 1% (vol/vol) tetrazolium dye. Phages are added to each well, and plates are incubated in an OmniLog system (Biolog, Inc., Hayward, Calif.) at 37° C. overnight. The tetrazolium dye indirectly measures the respiration of the bacterial cells. Respiration causes reduction of the tetrazolium dye. resulting in a color change to purple. The color intensity of each well is quantified as relative units of bacterial growth. For host infectivity determination, bacteria are inoculated at 10⁵ CFU per well and phages are added at a concentration of 10⁵ PFU per well for a multiplicity of infection (MOI) of 10. For cocktail synergy studies, bacteria are inoculated at 10⁶ CFU per well and phages are added at a concentration of 10⁵ PFU per well for an MOI of 100.

### Example 11. Efficiency of Plating for Phages on the Host Strain

To determine how well each phage infects its original host strain, a dilution series spot plate assay is used to observe plaque formation according to techniques known in the art (21, 22). 50 Ed of a culture grown to the appropriate OD_{MK}, of each P. aeruginosa strain is used to individually inoculate 5 ml of molten top agar tempered to 55° C. The inoculated agar is mixed thoroughly and then is spread over square LB agar plates. Top agar is allowed to set for approximately 45 min, at which time 4-μl aliquots of 10¹⁰ to 10² plague forming units (PFU) in 10-fold dilutions of each phage are spotted on the surface. Spots are allowed to fully absorb into the top agar, after which plates are incubated at 37° C. for 24 h and plaque formation is assessed.

### Example 12. Time-Kill Analysis of P. aeruginosa WT, and ΔlasIΔrhlI Δpqs4 Treated with Phages

Time-kill experiments are used to provide a quantitative analysis of phage bactericidal activity as described in Regeimbal et al. (21). Overnight culture of P. aeruginosa strains are diluted 1:1,000 in fresh LB broth to a final concentration of ˜1×10⁶ CFU per ml. Twenty-milliliter aliquots are then transferred to 250-ml Erlenmeyer flasks and incubated at 37° C. with shaking at 200 rpm for 2 h. Samples are then challenged with either 2×10¹¹ PFU per ml of phage cocktail or an equal volume of sterile phosphate-buffered saline (PBS) and are returned to incubation at 37° C. with shaking at 200 rpm. One-hundred-microliter aliquots are taken at 0, 2, 4, and 24 h, serially diluted in PBS, and are plated on LB agar. Plates are incubated at 37° C. for 24 h, and colonies are subsequently enumerated.

### Example 13. Propagation and Purification of Phage Strains

High-titer phage stocks for in vivo experimentation are propagated and amplified in corresponding host bacteria by standard procedures (21, 23). Large-scale phage preparations are purified by cesium chloride density centrifugation (24) and are filtered through a 0.22-μm filter (Millipore Corporation, Billerica, Mass.) prior to the treatment of animals. Phage stocks are stored at 4° C. indefinitely.

### Example 14. Murine Infection Assays

P. aeruginosa strains were grown on Pseudomonas Isolation Agar (PIA) for 16-18 h at 37° C. and suspended in PBS to an OD₆₀₀ of 0.5, corresponding to ˜10⁹ CFU/mL. Inocula were adjusted spectrophotometrically to obtain the desired challenge dose in a volume of 50 μL. Six week old female Balb/c mice (Jackson Laboratories, Bar Harbor, Me.) were anesthetized by i.p. injection of 0.2 mL of a mixture of ketamine (25 mg/mL) and xylaxine (12 mg/mL). Groups of four mice were infected by non-invasive intratracheal instillation of dilutions of P. aeruginosa UCBPP-PA14 P1-lux or isogenic QS mutants (25). Mice were observed over 5 days, and animals that succumbed to infection or appeared to be under acute distress were humanely euthanized and were included in the experiment results. The dose of each strain causing 50% lethality (LD₅₀) was calculated using the method of Reed and Muench. LD₅₀ values are reported as the negative log 10 of that dilution. Survival data were analyzed as Kaplan-Meier curves. Data were analyzed by the log rank test.

For colonization of mice, P. aeruginosa strains were grown and prepared as described above. Six-week-old female Balb/c mice were anesthetized and infected with sublethal doses (˜0.5 LD₅₀) of P. aeruginosa UCBPP-PA14 P1-lux or isogenic QS mutants as described in the preceding section. Mice were euthanized at 24 and 48 h post-infection and whole lungs were collected aseptically, weighed, and homogenized in 1 mL of PBS. Tissue homogenates were serially diluted and plated on PIA and CFU determination was made 16-18 h later. Comparison of the numbers of viable bacteria obtained in lung homogenates relied on the Kruskal-Wallis test for comparison of three groups or the Mann-Whitney Utest for two group analyses.

An additional group of animals was included for each P. aeruginosa strain examined for real time monitoring of colonization and localization of bioluminescent bacteria using an IVIS-Lumina LT III imaging system (PerkinElmer). Briefly, each group of mice was anesthetized with 3% isoflurane using an XGI-8 Gas Anesthesia System (Caliper Life Sciences), and imaged using medium binning, f/stop 1, subject height 1.5 cm. Images were acquired with up to 5 min exposure. Total photon emission from the ventral and dorsal sides of imaged mice was quantified using Living Image Software v4.0x (Xenogen Corp.). Statistical analyses were performed using GraphPad Prism software.

### Results

RhlR has recently been shown to function independently of RhlI in non-mammalian model systems. The finding that RhlR functioned in the absence of RhlI suggested that there could be RhlI-independent RhlR function during infection in mammals. The roles of the different QS regulators in a Balb/c murine model of acute P. aeruginosa lung infection were examined, and the LD₅₀ for WT, ΔrhlR, ΔrhlI, ΔlasR, and ΔlasI P. aeruginosa UCBPP-PA14 strains were determined. All of the strains carried a constitutively-expressed luxCDABE operon inserted in the chromosome at the glmS locus, which enabled monitoring of the time-dependent growth and location of bacterial cells in the host during the course of infection. The LD₅₀ of the WT was 1.9×10⁶. The ΔrhlR, ΔlasR, and ΔlasI mutants had 1.5-2.5-fold higher LD₅₀s than WT whereas the ΔrhlI mutant had an LD₅₀ 1.7-fold lower than the WT. Thus, the ΔrhlR, ΔlasR, and ΔlasI mutants all trended toward being attenuated for lethal dose while the ΔrhII mutant displayed the WT level of lethality in this mouse model of infection. LasR and LasI have been implicated in pathogenicity in rodents (26).

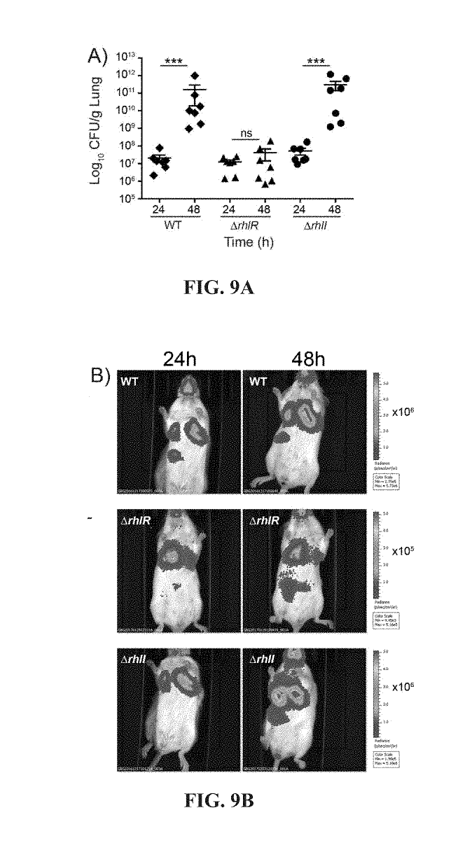

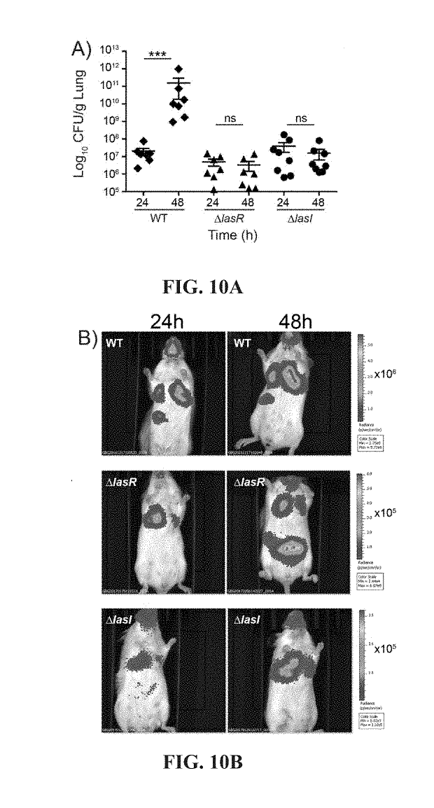

The time-dependence of the P. aeruginosa mouse infections were followed after intratracheal challenge with a sublethal dose (˜0.5 LD₅₀) of the strains under study. Colonization was monitored using an IVIS Imaging System to detect the constitutively produced luciferase from each P. aeruginosa strain. At 24 h, comparable levels of bioluminescence were detected, primarily in the lungs, in all of the infected mice. At 48 h, however, the signal was approximately 20-fold stronger in mice infected with the WT and the ΔrhlI strain than in mice infected with the ΔrhlR, ΔlasI, and ΔlasR strains (FIG. 9B and FIG. 10B). The imaging data were validated by determining the viable P. aeruginosa CFU per gram of lung homogenate. At 24 h post-infection, all of the infected mice had similar bacterial burdens. The average CFU/gram was 2.1×10⁷ for WT, 1.2×10⁷ for the ΔrhlR strain and 5.2×10⁷ for the ΔrhlI strain. At 48 h post-infection, the bacterial load in the lungs of mice infected with the WT and ΔrhlI mutant had increased to 1.6×10¹¹ and 3.1×10¹¹ CFUs, respectively. By contrast, the bacterial load in lungs of mice infected with the ΔrhlR strain did not increase significantly between 24 and 48 h, maintaining an average of 4.1×10⁷ CFU/gram. Finally, in the murine model of acute lung infection, the ΔlasI and ΔlasR mutants did not display the same level of virulence as the WT. At 48 h, mice infected with the ΔlasI and ΔlasR strains maintained the same bacterial load as at 24 h. (FIGS. 9A and 10A). This result supports the conclusion that, unlike RhlR/RhlI, the LasR/LasI components are an obligate pair during infection. Presumably, a ligand other than C4-HSL promoted RhlR function.

### Example 15. Murine Infection Assays

Once baseline infectivity of bacterial strains is established in the 48 hour infection model, the effect of phage on progression of infection is tested. Animals are infected with the WT and deletion strains of P. aeruginosa as described above in Example 14. Phage cocktails specifically targeted to the infection strain, selected and produced as described above (see, e.g., Examples 10-13) are dosed IP in sterile PBS over a range of concentrations determined by their MIC50 values (determined as pools in the time kill analysis at 24 hours, and dosed at a range of concentrations (e.g. MIC20, 50, and 90) using a total murine blood volume average of 1.5 mL/25 gm mouse).

Endpoints are as described above, including CFUs derived from aseptic lung homogenates and IVIS determinations of bioluminescence in whole animals. Efficacy is determined by plotting % CFU remaining versus dose at 48 hours post infection.

Additionally, QS inhibitors are tested in vivo for synergy with phage therapy against a WT strain of P. aeruginosa. In this experimental paradigm, WT P. aeruginosa is used, alone and in combination with the phage cocktail efficacious against the ΔrhlR strain. In this example, the ΔrhlR directed phage are selected to insure that phage infection is not dependent on QS-controlled gene expression. Experiments are carried out as described above, with intratracheal infection of P. aeruginosa, IP dosing of lytic phage cocktail, and QS inhibitor test articles (e.g., chrysin) introduced IV to achieve a steady state plasma concentration >ED90 in an otherwise inert vehicle. Endpoints are as described above, including CFUs derived from aseptic lung homogenates and IVIS determinations of bioluminescence in whole animals. Efficacy is determined by plotting % remaining CFU versus phage dose at constant drug concentration at 48 hours post infection.

### REFERENCES

1. Donlan R M and Costerton J W (2002) Biofilms: Survival mechanisms of clinically relevant microorganisms. Clin Microbiol Rev 15(2):167-193.
2. Hall-Stoodley L, et al. (2012) Towards diagnostic guidelines for biofilm-associated infections. FEMS Immunol Med Microbiol 65(2):127-145.
3. Kolter R and Greenberg E P (2006) Microbial sciences: The superficial life of microbes. Nature 441(7091):300-302.
4. Branda S S, et al., (2005) Biofilms: The matrix revisited. Trends Microbiol 13(1):20-26.
5. Flemming H-C and Wingender J (2010) The biofilm matrix. Nat Rev Microbiol 8(9): 623-633.
6. Escudie R, et al., (2011) Control of start-up and operation of anaerobic biofilm reactors: A overview of 15 years of research. Water Res 45(1):1-10.
7. Mah T F, et al. (2003) A genetic basis for Pseudomonas aeruginosa biofilm antibiotic resistance. Nature 426(6964):306-310.
8. Nguyen D, et al. (2011) Active starvation responses mediate antibiotic tolerance in biofilms and nutrient-limited bacteria. Science 334(6058):982-986.
9. Whiteley M, et al. (2001) Gene expression in Pseudomonas aeruginosa biofilms. Nature 413(6858):860-864.
10. Folsom J P, et al. (2010) Physiology of Pseudomonas aeruginosa in biofilms as revealed by transcriptome analysis. BMC Microbiol 10:294.
11. Dotsch A, et al. (2012) The Pseudomonas aeruginosa transcriptome in planktonic cultures and static biofilms using RNA sequencing. PLoS ONE 7(2):e31092.
12. Teal T K, et al., (2006) Spatiometabolic stratification of Shewanella oneidensis biofilms. Appl Environ Microbiol 72(11):7324-7330.
13. Rani S A, et al. (2007) Spatial patterns of DNA replication, protein synthesis, and oxygen concentration within bacterial biofilms reveal diverse physiological states. J Bacteriol 189(11):4223-4233.
14. Nadell C D, et al. (2010) Emergence of spatial structure in cell groups and the evolution of cooperation. PLOS Comput Biol 6(3):e1000716.
15. Picioreanu C, et al. (1998) Mathematical modeling of biofilm structure with a hybrid differential-discrete cellular automaton approach. Biotechnol Bioeng 58(1):101-116.
16. Hoyland-Kroghsbo et al. (2016) Quorum sensing controls the Pseudomonas aeruginosa CRISPR-Cas adaptive immune system PNAS 114(1): 131-135.
17. Gibson D G, et al. (2009) Enzymatic assembly of DNA molecules up to several hundred kilobases. Nature Methods 6(5):343-345.
18. Rietsch A, et al. (2005) ExsE, a secreted regulator of type III secretion genes in Pseudomonas aeruginosa. Proc Natl Acad Sci USA 102(22):8006-8011.
19. Choi et al. (2006) A 10-min method for preparation of highly electrocompetent Pseudomonas aeruginosa cells: Application for DNA fragment transfer between chromosomes and plasmid transformation. J. Microbiol. Methods 64: 391-397.
20. Damron et al. (2013) Construction of mobilizable mini-Tn7 vectors for bioluminescent detection of gram-negative bacteria and single-copy promoter lux reporter analysis. Appl. Environ. Microbiol. 79: 4149-4153.
20. Henry et al., (2012) Development of a high throughput assay for indirectly measuring phage growth using the OmniLog™ system. Bacteriophage (2012) 2:159-167.
21. Regeimbal et al. (2016) Personalized therapeutic cocktail of wild environmental phages rescues mice from Acinetobacter baumannii wound infections. Antimicrob Agents Chemother (2016) 60:5806-5816.
22. Sambrook et al. Molecular cloning: a laboratory manual, 2nd ed. Cold Spring Harbor Press, Cold Spring Harbor, N.Y., 1989.
23. Merril et al. (1996) Long-circulating bacteriophage as antibacterial agents. PNAS 93: 3188-3192.
24. Biswas et al., (2002) Bacteriophage therapy rescues mice bacteremic from a clinical isolate of vancomycin-resistant Enterococcus faecium. Infect Immun 70:204-210.
25. Revelli et al., (2012) A non-invasive intratracheal inoculation method for the study of pulmonary melioidosis Front. Cell. Infect. Microbiol. 2:164.
26. Tang et al., (1996) Contribution of Specific Pseudomonas aeruginosa Virulence Factors to Pathogenesis of Pneumonia in a Neonatal Mouse Model of Infection. Infect Immun 64: 37-43.

What is claimed is:

1. A composition comprising a quorum sensing (QS) inhibitor or agonist and a lytic phage.

2. The composition of claim 1, wherein the QS inhibitor is a compound of any one of Formulas I-VII.

3. The composition of claim 1, wherein the QS inhibitor or agonist is a compound in Tables 1-3.

4. The composition of claim 1, wherein the QS inhibitor or agonist is a small molecule.

5. The composition of claim 1, wherein the QS inhibitor binds to LasR or RhlR.

6. The composition of claim 1, wherein the lytic phage is selected from Table 4.

7. The composition of claim 1, wherein the lytic phage infects a particular microorganism.

8. The composition of claim 1, further comprising a plurality of lytic phages.

9. Use of the composition of claim 1 to inhibit QS, biofilm formation, biofilm streamer formation, virulence factor production, and/or an infection caused by a microorganism.

10. Use of a composition comprising a molecule of Formula I or listed in Tables 1-3 to inhibit QS, biofilm formation, biofilm streamer formation, virulence factor production, and/or infections caused by a microorganism.

11. The use of claim 9, further comprising administering an antibiotic.

12. The use of claim 9, wherein the microorganism is selected from bacteria and/or archaea.

13. The use of claim 9, wherein the microorganism is a pathogenic bacterium.

14. The use of claim 9, wherein the bacterium is resistant to at least one antibiotic.

15. The use of claim 9, wherein the bacteria is selected from Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anabaena, Anabaenopsis, Anaerobospirillum, Anaerorhabdus, Aphanizomenon, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Bordetella, Borrelia, Brachyspira, Branhamella, Brevibacillus, Brevibacterium, Brevundimonas, Brucella, Burkholderia, Buttiauxella, Butyrivibrio, Calymmatobacterium, Camesiphon, Campylobacter, Capnocytophaga, Capnylophaga, Cardiobacterium, Catonella, Cedecea, Cellulomonas, Centipeda, Chlamydia, Chlamydophila, Chromobacterium, Chryseomonas, Chyseobacterium, Citrobacter, Clostridium, Collinsella, Comamonas, Corynebacterium, Coxiella, Cryptobacterium, Cyanobacteria, Cylindrospermopsis, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gloeobacter, Gordona, Haemophilus, Hafnia, Hapalosiphon, Helicobacter, Helococcus, Hemophilus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptospirae, Leptotrichia, Leuconostoc, Listeria, Listonella, Lyngbya, Megasphaera, Methylobacterium, Microbacterium, Micrococcus, Microcystis, Mitsuokella, Mobiluncus, Moellerella, Moraxella, Morganella, Mycobacterium, Mycoplasma, Myroides, Neisseria, Nocardia, Nocardiopsis, Nodularia, Nostoc, Ochrobactrum, Oeskovia, Oligella, Orientia, Paenibacillus, Pantoea, Parachlamydia, Pasteurella, Pediococcus, Peptococcus, Peptostreptococcus, Phormidium, Photobacterium, Photorhabdus, Phyllobacterium, Phytoplasma, Planktothrix, Plesiomonas, Porphyromonas, Prevotella, Propionibacterium, Proteus, Providencia, Pseudoanabaena, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Schizothrix, Selenomonas, Serpulina, Serratia, Shewenella, Shigella, Simkania, Slackia, Sphaerotilus, Sphingobacterium, Sphingomonas, Spirillum, Spiroplasma, Spirulina, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Trichodesmium, Tropheryma, Tsakamurella, Turicella, Umezakia, Ureaplasma, Vagococcus, Veillonella, Vibrio, Weeksella, Wolinella, Xanthomonas, Xenorhabdus, Yersinia, Yokenella. Acinetobacter baumannii, Actinobacillus actinomycetemcomitans, Actinobacillus pleuropneumoniae, Actinomyces bovis, Actinomyces israelii, Bacillus anthracis, Bacillus ceretus, Bacillus coagulans, Bacillus liquefaciens, Bacillus popillae, Bacillus subtilis, Bacillus thuringiensis, Bacteroides distasonis, Bacteroides fragilis, Bacteroides thetaiotaomicron, Bacteroides vulgatus, Bartonella bacilliformis, Bartonella Quintana, Beneckea parahaemolytica, Bordetella bronchiseptica, Bordetella parapertussis, Bordetella pertussis, Borelia burgdorferi, Brevibacterium lactofermentum, Brucella abortus, Brucella canis, Brucella melitensis, Brucella suis, Burkholderia cepacia, Burkholderia mallei, Burkholderia pseudomallei, Campylobacter fetus, Campylobacter jejuni, Campylobacter pylori, Cardiobacterium hominis, Chlamydia pneumoniae, Chlamydia psittaci, Chlamydia trachomatis, Chlamydophila abortus, Chlamydophila caviae, Chlamydophila felis, Chlamydophila pneumonia, Chlamydophila psittaci, Chryseobacterium eningosepticum, Clostridium botulinum, Clostridium butyricum, Clostridium coccoides, Clostridium dijficile, Clostridium leptum, Clostridium tetani, Corynebacterium xerosis, Cowdria ruminantium, Coxiella burnetii, Edwardsiella tarda, Ehrlichia sennetsu, Eikenella corrodens, Elizabethkingia meningoseptica, Enterobacter aerogenes, Enterobacter cloacae, Enterococcus faecalis, Escherichia coli, Escherichia hirae, Flavobacterium meningosepticum, Fluoribacter bozemanae, Francisella tularensis, Francisella tularensis biovar Tularensis, Francisella tularensis subsp. Holarctica, Francisella tularensis subsp. nearctica, Francisella tularensis subsp. Tularensis, Francisella tularensis var. palaearctica, Fudobascterium nucleatum, Fusobacterium necrophorum, Haemophilus ducreyi, Haemophilus influenzae, Helicobacter pylori, Kingella kingae, Klebsiella mobilis, Klebsiella oxytoca, Klebsiella pneumoniae, Lactobacillus acidophilus, Lactobacillus casei, Lactobacillus hilgardii, Lactobacillus pentosus, Lactobacillus plantarum, Lactobacillus rhamnosus, Lactococcus lactis, Legionella bozemanae corrig., Legionella pneumophila, Leptospira alexanderi, Leptospira borgpetersenii, Leptospira fainei, Leptospira inadai, Leptospira interrogans, Leptospira kirschneri, Leptospira noguchii, Leptospira santarosai, Leptospira weilii, Leuconostoc lactis, Leuconostoc oenos, Listeria ivanovii, Listeria monocytogenes, Moraxella catarrhalis, Morganella morganii, Mycobacterium africanum, Mycobacterium avium, Mycobacterium avium subspecies paratuberculosis, Mycobacterium bovis, Mycobacterium bovis strain BCG, Mycobacterium intracellulare, Mycobacterium kansasii, Mycobacterium leprae, Mycobacterium marinum, Mycobacterium tuberculosis, Mycobacterium typhimurium, Mycobacterium ulcerans, Mycoplasma hominis, Mycoplasma mycoides, Mycoplasma pneumoniae, Neisseria gonorrhoeae, Neisseria meningitidis, Neorickettsia sennetsu, Nocardia asteroides, Orientia tsutsugamushi, Pasteurella haemolytica, Pasteurella multocida, Plesiomonas shigelloides, Propionibacterium acnes, Proteus mirabilis, Proteus morganii, Proteus penneri, Proteus rettgeri, Proteus vulgaris, Providencia alcalifaciens, Providencia rettgeri, Pseudomonas aeruginosa, Pseudomonas mallei, Pseudomonas pseudomallei, Pyrococcus abyssi, Rickettsia akari, Rickettsia canadensis, Rickettsia canadensis corrig, Rickettsia conorii, Rickettsia montanensis, Rickettsia montanensis corrig, Rickettsia prowazekii, Rickettsia rickettsii, Rickettsia sennetsu, Rickettsia tsutsugamushi, Rickettsia typhi, Rochalimaea quintana, Salmonella arizonae, Salmonella choleraesuis subsp. arizonae, Salmonella enterica subsp. Arizonae, Salmonella enteritidis, Salmonella paratyphi, Salmonella typhi, Salmonella typhimurium, Selenomonas nominantium, Selenomonas ruminatium, Serratia marcescens, Shigella boydii, Shigella dysenteriae, Shigella flexneri, Shigella sonnei, Spirillum minus, Staphylococcus aureus, Staphylococcus epidermidis, Staphylococcus equi, Staphylococcus lugdunensis, Stenotrophomonas maltophila, Streptobacillus moniliformis, Streptococcus agalactiae, Streptococcus bovis, Streptococcus ferus, Streptococcus pneumoniae, Streptococcus pyogenes, Streptococcus viridans, Streptomyces ghanaenis, Streptomyces hygroscopicus, Streptomyces phaechromogenes, Treponema carateum, Treponema denticola, Treponema pallidum, Treponema pertenue, Vibrio cholerae, Vibrio parahaemolyticus, Vibrio vulnificus, Xanthomonas maltophilia, Yersinia enterocolitica, Yersinia pestis, Yersinia pseudotuberculosis, Zymomonas mobilis, or Fusospirochetes.

16. The use of claim 9 to treat infections associated with P. aeruginosa, including respiratory infections associated with cystic fibrosis, infections associated with burns, and urinary tract infections.

17. The use of claim 9 to treat sepsis, bacterial endocarditis, pneumonia, respiratory infections, particularly in patients with cystic fibrosis and COPD, urethritis and cervicitis, a urinary tract infection, prostatitis, cystitis, infectious kidney stones, sinusitus, otitis media, tonsillitis, mastoiditis, periodontal disease, dental caries, gingivitis, periodontitis, breath malodor, Gram-negative infections, Gram-positive infections, bronchiectasis, osteomyelitis, Legionnaire's disease, an accumulation of biofilm in the lungs or digestive tract, emphysema, chronic bronchitis, bronchitis, skin infections, soft tissue infections, rosacea, acne, dermatitis, ulcers from peripheral vascular disease, a burn injury, and trauma.

18. The use of claim 9 to treat pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), S. pyogenes, S. agalactiae, Streptococcal groups C-F (minute-colony streptococci), viridans streptococci, Corynebacterium spp., Clostridium spp., or Bartonella henselae; uncomplicated acute urinary tract infections related to infection by S. saprophyticus or Enterococcus spp.; sexually transmitted diseases related to infection by Chlamydia trachomatis, Haemophilus ducreyi, Treponema pallidum, Ureaplasma urealyticum, or Nesseria gonorrheae; toxin diseases related to infection by S. aureus (food poisoning and Toxic shock syndrome), or Groups A, S, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by C. trachomatis, N. gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; odontogenic infection related to infection by viridans streptococci; persistent cough related to infection by Bordetella pertussis; gas gangrene related to infection by Clostridium perfringens or Bacteroides spp.; skin infection by S. aureus, Propionibacterium acne; atherosclerosis related to infection by Helicobacter pylori or Chlamydia pneumoniae; or the like.

19. The use of claim 9, to treat, disinfect or sterilize implanted/indwelling medical devices in order to prevent infections from said medical devices, including biliary stent infections, orthopedic implant infections, and catheter-related infections, and to treat, disinfect or sterilize industrial manufacturing facilities, hospitals, and/or equipment used with surgical procedures.

20. The use of claim 9, to treat, disinfect or sterilize both plant and animal based food products, as well as inhibit bacterial growth and contamination in animal and plant food processing, handling, packaging, production and preservation.

21. The use of claim 9, to prevent or treat infections in animals.

22. The use of claim 9 to prevent or treat biofilm formation on the bottom of ships, to prevent clogging from bacterial contamination in machinery or medical devices, or to prevent or treat infection associated with food supplies.

23. A method of screening for small molecule inhibitors of LasR or RhlR comprising:
(1) inducing expression of LasR or RhlR in the presence of an autoinducer (AI) to form a LasR or Rhl/AI complex; and
(2) measuring the expression levels of a reporter under the control of a LasR or RhlR promoter, wherein expression of the reporter occurs upon binding of the LasR or RhlR/AI complex to the LasR or RhlR promoter.

24. The method of claim 23, wherein arabinose is used to induce expression of LasR or RhlR.

25. The method of claim 23, wherein the reporter comprises luciferase.
